(19) United States
(12) Patent Application Publication
(10) Pub. No.: US 2006/0240462 A1
(43) Pub. Date: Oct. 26, 2006

(54) Methods for amplification and detection of nucleic acids

(75) Inventors: Alison Velyian Todd, Glebe (AU); Tanya Lynn Applegate, Greenwich (AU); Tram Bich Doan, Fairfield West (AU); Paul Ean Young, Engadine (AU)

Correspondence Address: WOODCOCK WASHBURN LLP, ONE LIBERTY PLACE, 46TH FLOOR, PHILADELPHIA, PA, 19103, US

(73) Assignee: Johnson & Johnson Research Pty Limited, Australian Technology Park (AU)

(21) Appl. No.: 11/409,369

(22) Filed: Apr. 21, 2006

Related U.S. Application Data
(60) Provisional application No. 60/673,633, filed on Apr. 21, 2005.

(51) Int. Cl. C12Q 1/68; C12P 19/34

(52) U.S. Cl. 435/6; 435/91.2

(57) ABSTRACT

Provided herein are methods for the combined amplification and detection of one or a plurality of target nucleic acid molecules. The methods encompass the use of an antisense strand of a catalytic nucleic acid in a primer for amplification such that an amplicon produced thereby includes an active catalytic nucleic acid capable of indicating the presence of the target sequences through the modification of a reporter substrate. Devices and kits are also provided. DNA molecules for practicing the methods are also provided. The DNA molecules comprise at least a first portion complementary to at least a first portion of a target nucleic acid sequence, a second portion complementary to an antisense sequence of a second portion of the target nucleic acid sequence, and a third portion comprising an antisense sequence of a catalytic nucleic acid; the third portion positioned between the first and second portions of said DNA molecule.

### CROSS-REFERENCE TO RELATED APPLICATIONS

This claims priority to U.S. Provisional Application No. 60/673,633, filed Apr. 21, 2005, the entirety of which is herein incorporated by reference.

### FIELD OF THE INVENTION

This relates to methods of amplifying, detecting, and quantifying nucleic acid molecules in a sample. More particularly, it relates to such methods wherein the amplification is substantially isothermal. It also relates to kits and devices for implementing such methods.

### BACKGROUND OF THE INVENTION

Various publications, which may include patents, published applications, technical articles and scholarly articles, are cited throughout the specification in parentheses, and full citations of each may be found at the end of the specification. Each of these cited publications is incorporated by reference herein, in its entirety.

Analysis of nucleic acids often involves small samples, with minute quantities of nucleic acid, and even more minute quantities of the analyte (nucleic acid sequence) of interest. Methods for analyzing nucleic acids by first amplifying nucleic acid sequences in vitro through the use of enzymes, such as DNA and RNA polymerases, are known in biotechnology. These methods typically require detailed and sometimes difficult analysis after nonselective amplification of the nucleic acids in a sample. Catalytic nucleic acid enzymes, such as DNAzymes and ribozymes, that can modify substrates, for example reporter substrates, are also used for analysis of nucleic acids, however they lack the ability to amplify the target of interest in a sample.

Nucleic acid amplification techniques mediated by DNA polymerases include the well-known polymerase chain reaction (“PCR”) (See e.g. U.S. Pat. Nos. 4,683,202, 4,683,195, 4,000,159, 4,965,188, and 5,176,995; see also Chehab et al., 1987; Saiki et al., 1985). Other DNA polymerase-based methods include strand displacement amplification (“SDA”) (Walker et al., 1992), and rolling circle amplification (“RCA”) (Lizardi et al., 1998). More recently developed was loop-mediated isothermal amplification (“LAMP”) (Notomi et al., 2000; Nagamine et al., 2002). Still other techniques for amplification of nucleic acid are mediated by RNA polymerase and include techniques such as transcription-mediated amplification (“TMA”) (Jonas et al., 1993), self-sustained sequence replication (“SSSR” or “3SR”) (Fahy et al., 1991) and nucleic acid sequence replication-based amplification (“NASBA”) (Compton, 1991).

In addition to the techniques for amplification of nucleic acids such as those described above, there are other strategies used with nucleic acids. For example, some involve amplification of a detection signal to increase sensitivity rather than, or in addition to, amplification of the nucleic acid target, such as through the use of a reporter. For example, the Branched DNA assay (Urdea et al., 1993) biochemically amplifies a detection signal by employing a secondary reporter molecule (e.g. alkaline phosphatase). Fluorescence correlation spectroscopy (FCS) employs electronic amplification of a detection signal to enhance sensitivity (Eigen & Rigler, 1994).

Several methods allow combined target amplification and detection in a closed system, i.e., in a single reaction vessel. These methods include the Molecular Beacon (Tyagi and Kramer, 1996), Taqman™ (Lee et al., 1993), and HybProbe assays (Wittwer et al., 1997) all of which depend on internal hybridization probes, as well as the Sunrise™ (Nazarenko et al., 1997) and DzyNA assays (WO99/45146 and Todd et al., 2000) which each utilize modified primers. These combined amplification and detection approaches have all been used to detect the amplification products of PCR. Some have also been used with other amplification technologies. For example, Molecular Beacon probes have been used to detect amplification products of NASBA (Leone et al., 1998) and SDA (Vet et al., 2002).

Homogeneous single-tube assays have several advantages over methods that separately analyze amplicons post amplification. Such closed or sealed-tube methods are faster and simpler because they require fewer manipulations. A closed system also eliminates any potential for false positives associated with contamination by amplicons from prior reactions. Homogeneous reactions can preferably be monitored in real time where changes in the signal intensity reflect amplification of specific target sequence(s) present in the sample.

Unlike methods which separately amplify either the amount of target nucleic acid or the detection signal, catalytic nucleic acids have been used in combination with in vitro amplification protocols as a means of generating signal and allowing real-time monitoring of the amplification of nucleic acid target sequences (Todd et al., 2000; U.S. Pat. No. 6,140,055; U.S. Pat. No. 6,201,113; WO 99/45146; PCT/IB99/00848; WO 99/50452). The zymogene or “DzyNA” approach concurrently amplifies both target nucleic acid sequence and signal (U.S. Pat. No. 6,140,055; U.S. Pat. No. 6,201,113; WO 99/45146, Todd et al., 2000). This is possible because a catalytic DNAzyme or ribozyme is co-amplified along with target nucleic acid sequence(s). The co-amplified catalytic nucleic acid sequences then function as true catalytic “enzymes” capable of multiple turnover. As such, each catalytic nucleic acid amplified cleaves multiple reporter substrates, producing an amplified signal. The DzyNA strategy is compatible with amplification strategies that include PCR (also known as “zymogene” PCR), SDA, RCA and TMA/NASBA (WO9945146, Todd et al., 2000, Singh et al., 2004).

The available methods for analyzing nucleic acids provide certain advantages and disadvantages. For example, PCR requires thermocycling and thus requires more complex (and expensive) apparatus than isothermal techniques such as SDA, TMA and LAMP protocols.

There is also a tradeoff between the primer requirements and the specificity of the amplification. LAMP, for example, is a rapid amplification method that provides high specificity since it requires 4 or more primers to recognise 6 or more sequences within each target sequence to be amplified. In comparison SDA uses 4 primers to recognise 4 regions of sequence in a target, while PCR uses only 2 primers to recognise 2 target sequence regions.

Additional specificity can be achieved when cleavage or hybridisation of internal target-specific probes are monitored in real time. Methods for accomplishing this include Molecular Beacon (Tyagi and Kramer, 1996), Taqman™ (Lee et al., 1993), and HybProbe assays (Wittwer et al., 1997). The TaqMan PCR is widely used but is difficult to multiplex due to the high concentrations of primers used. DzyNA PCR, for example, allows generic multiplexing, but has the potential to produce signal from primer/dimer when primer design or reaction conditions are sub-optimal.

There is, therefore, a need in the art for methods that allow for amplification of both target nucleic acid sequences and related detection signals, are isothermal, have potentially relaxed primer requirements while maintaining high specificity, allow for multiplex detection of multiple targets in a single reaction vessel, can be conducted in a closed system, and monitored in real time. The present invention provides methods that meet these needs by employing amplification, for example using modified LAMP primers coupled with multiplex signal amplification using the DzyNA strategy in real time.

### SUMMARY OF THE INVENTION

In a first aspect, the invention provides methods for detecting the presence of a nucleic acid sequence in a sample. The methods comprise:

(a) providing a primer mixture comprising:
(i) a pair of inner primers; or
(ii) a pair of inner primers and at least one outer primer; or
(iii) a pair of inner primers and at least one loop primer; or
(iv) a pair of inner primers, at least one outer primer, and at least one loop primer;

wherein the pair of inner primers comprises a forward inner primer and a backward inner primer, and each said inner primer comprises a first portion that hybridizes to a sense sequence of a target nucleic acid sequence, and a second portion that hybridizes to an antisense sequence of the target nucleic acid sequence;

wherein each said outer primer hybridizes to a portion of the target nucleic acid sequence;

wherein each said loop primer comprises a portion complementary to a single stranded loop region on an amplicon produced from the extension of the forward inner primer or the backward inner primer;

wherein at least one primer in said primer mixture comprises an antisense sequence of a catalytic nucleic acid such that a corresponding sense strand of said catalytic nucleic acid is incorporated in an amplicon produced during amplification of said target nucleic acid sequence;

wherein, when the primer mixture does not comprise any loop primers, an antisense sequence of a catalytic nucleic acid is positioned between the first and the second portion of one or both of the forward and backward inner primers; and

wherein, when the primer mixture comprises at least one loop primer an antisense sequence of a catalytic nucleic acid is positioned either between the first and the second portion of one or both of the forward or backward inner primer; at the 5′ end of one or more loop primers, or both;

(b) contacting the sample with the primer mixture under conditions permitting catalytic nucleic acid activity and target-dependent, primer-initiated, DNA polymerase-mediated nucleic acid amplification;

wherein the DNA polymerase has strand displacement activity;

(c) incubating the sample with the primer mixture to allow the primer mixture to initiate amplification, when the target nucleic acid sequence is present, to produce amplicons comprising the catalytic nucleic acid; and

(d) determining the presence of the catalytic nucleic acid activity, thereby determining the presence of target nucleic acid sequence in the sample.

Also provided herein are methods of detecting the presence of each of a plurality of target nucleic acid sequences in a sample. The methods comprise:

(a) providing a primer mixture comprising, for each of the plurality of target nucleic acid sequences to be detected at least:
(i) a pair of inner primers; or
(ii) a pair of inner primers and at least one outer primer; or
(iii) a pair of inner primers and at least one loop primer; or
(iv) a pair of inner primers, at least one outer primer, and at least one loop primer;

wherein each pair of inner primers comprises a forward inner primer and a backward inner primer, and each said inner primer comprises a first portion that hybridizes to a sense sequence of at least one of the plurality of target nucleic acid sequences, and a second portion that hybridizes to an antisense sequence of that target nucleic acid sequence;

wherein each said outer primer hybridizes to a portion of at least one of the plurality of target nucleic acid sequences;

wherein each said loop primer comprises a portion complementary to a single stranded loop region on an amplicon produced from the extension of at least one forward inner primer or backward inner primer corresponding to at least one of the plurality of target nucleic acid sequences;

wherein for each of the plurality of target nucleic acid sequences, at least one primer in said primer mixture comprises an antisense sequence of a distinctly detectable catalytic nucleic acid such that a corresponding sense strand of said distinctly detectable catalytic nucleic acid is incorporated in an amplicon produced during amplification of that target nucleic acid sequence;

wherein for each of the plurality of target nucleic acid sequences, when the primer mixture does not comprise any loop primers for that target nucleic acid sequence, said antisense sequence of a distinctly detectable catalytic nucleic acid is positioned between the first and the second portion of one or both of the forward or backward inner primers; and

wherein for each of the plurality of target nucleic acid sequences, when the primer mixture comprises at least one loop primer for that target nucleic acid sequence, the antisense sequence of a distinctly detectable catalytic nucleic acid is positioned between the first and the second portion of one or both of the forward or backward inner primers, or at the 5′ end of one or more loop primers, or both;

(b) contacting the sample with the primer mixture under conditions permitting catalytic nucleic acid activity and target sequence-dependent, primer-initiated, DNA polymerase-mediated nucleic acid amplification;

wherein the DNA polymerase has strand displacement activity;

(c) incubating the sample with the primer mixture to allow the primer mixture to initiate amplification of each of the plurality of target nucleic acid sequences, when that target nucleic acid sequence is present, to produce amplicons comprising the distinctly detectable catalytic nucleic acid; and

(d) determining the presence of each of the uniquely distinctly detectable catalytic nucleic acid activities, thereby determining the presence of the corresponding target nucleic acid sequence in the sample.

In another aspect of the invention, methods are provided for detecting the presence of any of a plurality of target nucleic acid sequences in a sample. The methods comprise:

(a) providing a primer mixture comprising one or more primers sufficient for amplifying each of the plurality of target nucleic acid sequences to be detected;

wherein for each of the plurality of target nucleic acid sequences, there is at least one primer in said primer mixture comprising an antisense sequence of a catalytic nucleic acid such that a corresponding sense strand of said catalytic nucleic acid is incorporated into an amplicon produced during amplification of that target nucleic acid sequence;

(b) contacting the sample with the primer mixture under conditions permitting catalytic nucleic acid activity and target sequence-dependent, primer-initiated, DNA polymerase-mediated nucleic acid amplification;

(c) incubating the sample with the primer mixture to allow the primer mixture to initiate amplification of any of the plurality of target nucleic acid sequences, when that target nucleic acid sequence is present, to produce amplicons comprising the catalytic nucleic acid; and

(d) determining the presence of the catalytic nucleic acid activity from an amplicon produced during the amplification of any of the target nucleic acid sequences, thereby determining the presence of any of the target nucleic acid sequences in the sample.

Also provided herein are DNA molecules comprising at least a first portion complementary to at least a first portion of a target nucleic acid sequence, a second portion complementary to an antisense sequence of a second portion of the target nucleic acid sequence, and a third portion comprising an antisense sequence of a catalytic nucleic acid; said third portion positioned between the first and second portions of said DNA molecule.

Methods for the use of such DNA molecules are provided herein, as are kits comprising the novel DNA molecules. Kits are also provided for practicing the methods disclosed herein.

Also provided in a further aspect of the invention are devices for detecting the presence, in a sample placed therein, of at least one target nucleic acid sequence. The devices comprise:

a reaction vessel into which the sample is introduced, said reaction vessel comprising a reaction mixture suitable for target sequence-dependent, primer-initiated, DNA polymerase-mediated nucleic acid amplification under conditions also permitting catalytic nucleic acid activity,

the reaction mixture comprising the reactants for amplification of nucleic acids in the sample and a primer mixture comprising one or more primers sufficient for amplifying each of the at least one target nucleic acid sequences to be detected;

wherein for each of the at least one target nucleic acid sequences to be detected, there is at least one primer in said primer mixture comprising an antisense sequence of a catalytic nucleic acid such that a corresponding sense strand of said catalytic nucleic acid is incorporated into an amplicon produced when that target is present in the sample; said sense strand comprising an active catalytic nucleic acid that recognizes and modifies a corresponding substrate;

a support means for bearing the substrate for each catalytic nucleic acid activity corresponding to each of the at least one target nucleic acid sequences to be detected; wherein each such substrate produces a detectable signal upon modification thereof by the catalytic nucleic acid.

These and other aspects of the invention will become more with reference to the detailed description, figures, and working examples which are provided to illustrate various aspects of the invention. This disclosure is not intended to, and should not be construed to, limit the invention to that disclosed.

### BRIEF DESCRIPTION OF THE DRAWINGS

FIG. 1: Schematic of the primers used in standard and accelerated LAMP reactions.

Panel A: Depicts the position of primers in relation to a nucleic acid sequence to be amplified.

Panel B: The Inner Forward Primers (FIP) and Inner Backward Primers (BIP), which are essential to all LAMP protocols are shown. These primers contain regions that are either in the sense or the anti-sense (complement, “c”) orientation with respect to either the sense or anti-sense (complement, “c”) of target nucleic acid strands.

Panel C: Additional primers, which may be useful in reducing reaction times, are the outer Forward (F3), Outer Backward (B3), Forward Loop F or Backward Loop B primers are shown.

FIG. 2: Examples of primers useful in the methods provided herein. The preferred positions where an antisense sequence of a catalytic nucleic acid (cDz), such as a generic DNAzyme, can be placed are indicated. For example, for either the forward or backward inner primers, the cDz sequence can be included between the F1c and F2 sequence in the FIP primer (i) cDz/FIP, or between the B1c and B2 sequence in the BIP primer (ii) cDz/BIP. The cDz sequence can also be included 5′ to the loop B sequence in the loop primer (iii) cDz/Loop B, and/or 5′ to the loop F sequence in the loop primer (iv) cDz/Loop F.

FIG. 3: Example of “point of care” testing (e.g. a “dip stick”). This shows a generalised format of an exemplary multiplex “dipstick” test, which can be provided as a kit, as a useful application of the methods provided herein. This reaction can be performed under isothermal conditions at any temperature suited to the particular methods.

Step i: A multiplex reaction amplifies several targets of interest. If the reaction tube remains translucent to the unaided eye, this indicates no amplification has occurred (negative amplification result for all targets). If the reaction tube becomes turbid or cloudy, this indicates successful amplification (positive amplification result for one or more targets).

Step ii: The exact species present in the positive samples can then be identified by exposure to the dipstick.

Step iii: In this example, five dual-labeled fluorescent substrates are covalently attached to the dipstick, for example, at discrete locations. Five substrates can allow, for example, for the identification of one positive and one negative amplification control and three targets of interest. While the sequences of each substrate must be distinct from each other, the fluorophore/quencher dye pair or other detectable portion may be the same.

Step iv: Active DNAzymes generated from each target present in the sample will cleave the substrate corresponding to that target. Cleavage of each fluorescent substrate will result in removal of the quencher and concomitant fluorescence. For example, where the substrates are attached in discrete bands, the resulting banding pattern on the strip test could identify the target species in the test sample. Such a pattern could also be used, for example, as an indication for personalised therapy.

FIG. 4: Additional exemplary applications of devices for use herein.

(a): The “dipstick test” concept can be extended to a “Striptest”:

Step i: Amplification: Multiplex amplification of a panel of samples—each tube is used to amplify the one or more targets present in the sample,

Step ii: Amplicon transfer: The positive samples are transferred to the Striptest, which contains covalently-attached, single-colour capture substrates, specific to each amplicon, preferably in discrete locations;

Step iii: Detection: Target-specific amplification results in cleavage of the corresponding substrate with concomitant signal generation, preferably at a corresponding position. The results, preferably in a pattern of bands, identify specific targets in a panel of patient samples, such as for viral screening of blood products.

(b): Exemplifying a multiplex amplification and detection or quantification that can be carried out in a homogeneous single vessel such as a microtube or microwell format.

Step i: Single tube multiplexed reaction: Amplicons from each target amplified from a multiplex reaction harbour specific DNAzyme tag, which cleaves complementary substrate (Sub) labelled with distinct fluorophore (F).

Step ii: Detection: Successful target amplification from a multiplex reaction can be determined by the wavelength of the signal generated by specific substrate cleavage at the end of the reaction. The change in fluorescence from these multiplexed reactions can be monitored by end point or in real time.

FIG. 5: Fluorescent real time quantitation (R²=0.998) of lambda DNA (10⁷ to 10³ copies).

FIG. 6: Fluorescent real time quantitation (R²=0.990) of lambda DNA (10⁷ to 10² copies) in a background of 100 ng genomic DNA.

FIG. 7: Amplification efficiencies resulting from various ratios of cDzX/LoopB primer to Loop B primer, as indicated.

FIG. 8: Comparison of the amplification using two different embodiments: Model A (cDzX/Loop B/) and Model B (cDzX/BIP) plotted on log and linear scales.

### DETAILED DESCRIPTION OF ILLUSTRATIVE EMBODIMENTS

In several of its aspects, the invention provides methods which exploit the use of the activity of both protein and nucleic acid enzymes in a novel manner to provide sensitive and simple methods of detecting and/or quantifying a single nucleic acid sequence, or even a plurality of such sequences. The methods can be practiced in the most simple of forms, such as dipstick tests, or test strips, which are also provided herein. The methods can also be used in more sophisticated applications as provided herein, for example real-time monitoring. Kits for practicing the methods are also provided.

Definitions:

Catalytic Nucleic Acids

“Catalytic nucleic acid molecule”, “catalytic nucleic acid”, and “catalytic nucleic acid sequence” as used herein refer to a nucleic acid having catalytic activity. More particularly, the terms encompass any DNA molecule or DNA-containing molecule having catalytic activity (e.g. any “deoxyribozyme” or “DNAzyme”), as well as any RNA or RNA-containing molecule having catalytic activity (e.g. any “ribozyme”), and any such catalytic nucleic acid is suitable for use herein.

Catalytic nucleic acids such as ribozymes and DNAzymes recognize a substrate and catalyze its chemical modification and may be also be referred to herein as “enzymatic nucleic acids”. DNAzymes have been shown to be capable of cleaving both RNA (Breaker and Joyce, 1994; Santoro and Joyce, 1997) and DNA (Carmi et al., 1996) molecules. Similarly, ribozymes have been shown to be capable of cleaving both RNA (Haseloff and Gerlach, 1988) and DNA (Raillard and Joyce, 1996) molecules.

Specific DNAzymes and ribozymes that recognize distinct target nucleic acid sequences through Watson Crick base pairing, and cleave these sequences at specific locations, for example between particular pairs of bases, have been characterized and are suitable for use in accordance with the methods, kits and devices provided herein. A catalytic nucleic acid can only cleave a target nucleic acid sequence provided that target sequence meets minimum sequence requirements. The target sequence must be substantially complementary to the hybridizing arms of the catalytic nucleic acid and the target must contain a specific sequence at the site of cleavage. Examples of such sequence requirements at the cleavage site include the requirement for purine:pyrimidine ribonucleotides for cleavage by the 10:23 DNAzyme (Santoro and Joyce, 1997), and the requirement for the sequence uridine:X where X is A, C or U, but not G, for the hammerhead ribozymes (Perriman et al., 1992). The 10:23 and 8-17 DNAzymes are DNAzymes that are capable of cleaving nucleic acid substrates at specific RNA phosphodiester bonds (Santoro and Joyce, 1997). The 10:23 DNAzyme has a catalytic domain of 15 deoxynucleotides flanked by two substrate-recognition sites (referred to as domains or “arms”).

In the methods provided herein, the catalytic nucleic acids are used as a means of amplifying a detection signal to facilitate the specific identification of one or more target nucleic acid sequences. In particular, the antisense sequence of a catalytic nucleic is incorporated into a primer used in amplifying one or more target nucleic acid sequences. The amplification process produces amplicons that incorporate a sense strand of the catalytic nucleic acid (i.e the active DNAzyme or ribozyme) which can then be used to “report” the presence of the corresponding target nucleic acid sequence. A unique catalytic nucleic acid activity is used for each target nucleic acid where such sequences are to be individually detected. In some cases, the same catalytic nucleic acid activity may be used where individual detection of target nucleic acid sequences is not required, for example where an assay is designed merely to give a yes/no answer as to the presence of any of a plurality of target sequences. When the antisense sequence of a ribozyme is included in the primer, resulting in the production of amplicons comprising a ribozyme, an RNA polymerase and RNA polymerase promoter are required in the reaction system, and thus are deemed part of the conditions allowing amplification and detection even if not so stated.

The nucleic acid bases in the DNAzymes and ribozymes themselves can be the respective ribo- or deoxy-forms of the bases A, C, G, T, and U, as well as derivatives or analogs thereof. Many derivatives and analogs of these bases are known in the art. Examples of such derivatives are shown in Table 1.

TABLE 1
Nucleotide Base Analogs

| Abbreviation | Description |
|---|---|
| Ac4c | 4-acetylcytidine |
| chm5u | 5-(carboxyhydroxylmethyl)uridine |
| Cm | 2′-O-methylcytidine |
| cmnm5s2u | 5-carboxymethylaminomethyl thiouridine |
| D | Dihydrouridine |
| Fm | 2′-O-methylpseudouridine |
| Galq | β,D-galactosylqueosine |
| Gm | 2′-O-methylguanosine |
| L | Inosine |
| I6a | N6-isopentenyladenosine |
| M1a | 1-methyladenosine |
| M1f | 1-methylpseudouridine |
| M1g | 1-methy[guanosine |
| M11 | 1-methylinosine |
| M22g | 2,2-dimethylguanosine |
| M2a | 2-methyladenosine |
| M2g | 2-methylguanosine |
| M3c | 3-methylcytidine |
| M5c | 5-methylcytidine |
| M6a | N6-methyladenosine |
| M7g | 7-methylguanosine |
| mam5u | 5-methylaminomethyluridine |
| mam5s2u | 5-methoxyaminomethyl-2-thiouridine |
| Manq | β,D-mannosylmethyluridine |
| mcm5s2u | 5-methoxycarbonylmethyluridine |
| Mo5u | 5-methoxyuridine |
| Ms2i6a | 2-methylthio-N6-isopentenyladenosine |
| Ms2t6a | N-((9-β-ribofuranosyl-2-methylthiopurine-6- |
| | yl)carbamoyl)threonine |
| Mt6a | N-((9-β-ribofuranosylpurine-6-yl)N-methyl- |
| | carbamoyl)threonine |
| Mv | uridine-5-oxyacetic acid methylester |
| O5u | uridine-5-oxyacetic acid (v) |
| Osyw | Wybutoxosine |
| P | Pseudouridine |
| Q | Queosine |
| S2c | 2-thiocytidine |
| S2t | 5-methyl-2-thiouridine |
| S2u | 2-thiouridine |
| S4u | 4-thiouridine |
| T | 5-methyluridine |
| T6a | N-((9-β-D-ribofuranosylpurine-6-yl)carbamoyl)threoninetm |
| | 2′-O-methyl-5-methyluridine |
| Um | 2′-O-methyluridine |
| Yw | Wybutosine |
| X | 3-(3-amino-3-carboxypropyl)uridine, (acp3)u |
| araU | B,D-arabinosyl |
| araT | B,D-arabinosyl |

The skilled artisan will appreciate that such analogs can also be used to modify the oligonucleotides and primers herein, for example, in the synthesis of such molecules.

Amplification Methods:

“Amplification” of a target nucleic acid sequence, as used herein refers to copying of one or more target sequences. Preferably the amplification techniques used herein are in vitro techniques as discussed herein above. Methods of in vitro nucleic acid amplification based on DNA or RNA polymerase activity have many applications, for example, in disease diagnosis, forensics, and the study of genetics. Techniques for amplification of known nucleic acid sequences (“targets”) have been described. Methods of in vitro amplification include, but are not limited to, PCR, SDA, RCA, and LAMP, as well as TMA, NASBA, and 3SR.

Amplification products (“amplicons”) produced by PCR, SDA, RCA and LAMP are composed of DNA, whereas the amplicons produced by TMA, 3SR and NASBA are composed of RNA. The DNA or RNA amplicons generated by these methods can be used herein, for example, as markers of nucleic acid sequences associated with specific diseases or disorders. These amplification techniques allow the identification of changes which are quantitative (e.g. over expression, under expression, loss of heterozygosity, gene amplification) or qualitative (e.g. point mutations, translocations, deletions, insertions, relative presence or absence of a sequence).

LAMP Amplification

LAMP amplification is preferred for use in several aspects of the invention. LAMP rapidly amplifies DNA or RNA with high specificity and efficiency under isothermal conditions (Notomi et al., 2000; Nagamine et al., 2002; EP 1 020 534; EP 1 333 089; EP 1 327 679; EP 1 275 715). The first LAMP protocol published (Notomi et al., 2000) employed 4 primers, which recognise a total of 6 distinct sequences on a target DNA or cDNA to be amplified. This amplification is sometimes referred to herein as “standard LAMP.” The accelerated LAMP protocol employs 6 primers, which recognise a total of 8 sequences on the target nucleic acid (Nagamine et al., 2002; EP 1 327 679).

Several types of primers are used in standard LAMP. A pair of “outer” primers referred to as Forward (F) and backward (B) outer primers (also referred to as F3 and B3) of standard primer design is included. Also included is a pair of “inner” primers known as the forward and backward inner primers (also referred to as FIP and BIP). The inner primers each recognize two distinct regions relating to the target sequence. Each primer has one portion complementary to a part of the sense strand of the target. The inner primer also has a portion complementary to the anti-sense or complement of the target sequence. The terms “inner” primers, “FIP” or “BIP” primers and “sense/anti-sense” primers are equivalent. Additional primers, used in the accelerated LAMP protocol, are the forward and backward loop primers (loop F, loop B) which are complementary to the target.

LAMP is initiated when the target DNA is first copied using an inner primer. The copied DNA strand is displaced by the strand displacement activity of the DNA polymerase used in the amplification, and subsequently released following strand displacement mediated by a complementary strand being made from the corresponding outer primer. These single stranded sequences serve as template for further DNA synthesis using an inner primer that hybridizes to the other end of the target producing a stem-loop DNA structure. In subsequent rounds of the LAMP method, one inner primer hybridises to the loop on the product and initiates DNA synthesis, yielding the original stem-loop DNA and a new stem-loop with a stem twice as long. The final products are stem-loop DNAs with several inverted repeats of the target, and cauliflower-like structures with multiple loops formed by annealing between alternately inverted repeats of the target in the same strand. LAMP amplification is highly specific because it requires the primers to recognize each target DNA at six distinct sequences initially, and 4 or more distinct regions afterwards (Notomi et al., 2000; Nagamine et al., 2002).

The production of amplicons from a single target during LAMP can be measured in real time by monitoring turbidity or fluorescence (Mori et al., 2004; Iwanoto et al., 2003). During LAMP, by-product pyrophosphate ions bind to magnesium ions and form a white precipitate of magnesium pyrophosphate. An apparatus has been described that allows real-time analysis of the amplification of a single target during LAMP, by measuring changes in turbidity, (Mori et al., 2004). LAMP reactions can also be monitored in real time by measuring changes in fluorescence due to intercalation of SyBR green (Iwamoto et al., 2003) or ethidium bromide (EP 1 275 715 A1; Nagamine et al., 2002) into double-stranded LAMP amplicons. None of these methods of detection are amenable to multiplex real time analysis. LAMP is also incompatible with TaqMan™ probes because LAMP requires a strand displacing polymerase, whereas TaqMan™ requires a polymerase with 5′ exonuclease activity (Lee et al., 1993).

Additional sequences, such as restriction endonuclease recognition sequences, can be introduced into LAMP amplicons by inserting sequence between the sense and anti-sense regions of the inner FIP or BIP primers. Incubation with the correct restriction enzyme following LAMP resulted in the LAMP amplicons being reduced to the base unit so as to enable electrophoretic separation for confirmation of amplification of the target nucleic acid sequence (EP 1 020 534 A1; Iwamoto et al., 2003). A multistep protocol for using LAMP amplification for generating single-stranded nucleic acid suitable for hybridisation on chips required the sequential steps of digestion of LAMP amplicons with a restriction endonuclease, denaturation of the restriction endonuclease, primer extension reaction, and finally, exonuclease digestion. Insertion of an RNA polymerase promoter sequence, and a sequence encoding a ribozyme, has been suggested (EP 1 275 715 A1) as a means of generating RNA amplicons which could self cleave in cis.

As used herein a “substrate” or “chemical substrate” comprises any molecule which is recognized and modified by a catalytic nucleic acid molecule. A “reporter substrate” is a particular type of substrate which is preferred for use herein. “Reporter substrate” as used herein is a molecule which is both recognized and modified, e.g. cleaved, by a catalytic nucleic acid, and which also provides a facile means for measuring the cleavage, for example. Such measurement can be based on the decrease in the amount of the reporter substrate itself, or through the appearance of a readily measured or detected product, such as a cleavage product. As is exemplified herein, one preferred type of reporter substrate has a detectable signal molecule (e.g. a fluorescent marker) and a quencher of that signal in close proximity. Upon cleavage, physical separation of the quencher and the detectable signal occurs, resulting in a vast increase in detectable signal. The skilled artisan will appreciate that the terms “reporter substrate” and “substrate” are sometimes used synonymously herein, although the term “substrate” is technically broader than the term “reporter substrate,” as any useful reporter substrate for use herein must necessarily be a substrate for the catalytic nucleic acid of interest, e.g. it must be both recognized and cleaved.

“Modification” of a substrate, as used herein includes any chemical or physical change in a substrate. In preferred embodiments, “modifications” are made during the conversion of a substrate for a catalytic nucleic acid into the product of the reaction catalyzed. A wide range of such modifications is possible. In vitro evolution technology has facilitated the discovery of DNAzymes and ribozymes that are capable of catalyzing a broad range of reactions including cleavage (Breaker, 1997; Carmi et al., 1996; Raillard and Joyce, 1996; Santoro and Joyce, 1998) and ligation of nucleic acids (Cuenoud and Szostak, 1995), porphyrin metallation (Li and Sen, 1996), and the formation of carbon-carbon (Tarasow et al., 1997), ester (Illangasekare et al., 1995) or amide bonds (Lohse and Szostak, 1996). Therefore, it is possible to develop systems for detection of in vitro amplification products where the reporter substrate is a molecule other than a nucleic acid and/or the readout of the assay is dependent on a modification other than cleavage of a nucleic acid substrate.

Even where the substrate is a nucleic acid, it need not be a naturally-occurring nucleic acid. DNAzymes have been used in combination with PCR in a protocol that used chimeric DNA/RNA primers (WO 99/50452). These primers introduced purine:pyrimidine ribonucleotide residues into amplicons thus creating sites that are cleaved by DNAzymes provided target was present. Amplicons are cleaved in cis (i.e. by a catalytic nucleic acid on the same molecule) or in trans, depending on the particular embodiment. The protocol is sequence-specific and can distinguish even single-base differences. Furthermore, if the target sequence does not contain a natural purine:pyrimidine sequence, the cleavage site for the DNAzyme can be induced using mismatched primers (WO 99/50452, WO 99/45146) in the same way that mismatched primers have been used to induce artificial restriction enzyme (RE) sites (WO 99/50452, Todd, 1991).

“Target nucleic acid sequence” as used herein refers to a nucleic acid sequence of interest, for example, a nucleic acid sequence to be amplified, detected, or measured according to the methods herein, or to be amplified, detected, or measured through the use of the devices of the invention, or the kits of the invention. Target nucleic acid sequences, also referred to herein sometimes as “targets”, “target sequences”, “target nucleic acids”, or “target molecules” comprise a sequence that hybridizes with at least one primer when contacted therewith (e.g. under the conditions for amplification and detection), or is at least partially complementary to at least one primer. A target sequence can be either an entire molecule or a portion thereof. Also, it is to be understood that the use of the term “target nucleic acid sequence” with respect to detection of a particular trait does not necessarily mean that the target sequence must comprise or define the trait itself—i.e. in certain embodiments, the presence of the target sequence may be associated with a particular trait or quality, in other embodiments the trait or quality may be associated with the absence of the target sequence. For example a particular disease trait may be either associated with the presence of a mutated sequence, or with the absence of, or a decrease in wild-type sequence. Still other traits may be associated with an abundance or excess of a wild-type sequence. Similarly, in cases where a particular RNA or protein are encoded by a particular sequence, the target nucleic acid selection may either be in the coding or the noncoding strand of the corresponding DNA, for example, for reasons of preferred or convenient sites, such as recognition or cleavage sites within one or the other sequence. The skilled artisan will appreciate the assays and methods provided herein are flexible with respect to the design and selection of particular target nucleic acid sequences based on the particular application as well as the convenience or preference of the artisan developing the application.

“Primer” as used herein refers to a short segment of DNA or DNA-containing nucleic acid molecule, which (i) anneals under amplification conditions to a suitable portion of a DNA or RNA sequence to be amplified (e.g. a target sequence), and (ii) initiates extension, and is itself physically extended, via polymerase-mediated synthesis.

As with other primers, a “DzyNA primer” initiates extension, and is itself physically extended, via polymerase-mediated synthesis, moreover “DzyNA primer” refers to a nucleic acid sequence which contains both

a) sequences complementary to the target to be amplified (such that it anneals thereto under amplification conditions) and

b) the “anti-sense” (i.e. complementary) sequence of a catalytic nucleic acid molecule.

As used herein, the term “sense” strand or “sense” sequence with respect to nucleic acids or target nucleic acid sequences refers to a sequence that is in the nucleic acid or the target itself. It does not require that the reference strand encode a protein or an RNA, although the term is sometimes used in biotechnology to so indicate. Similarly, an “antisense” sequence would occur in the complement of the target, rather than in the target itself. In this context, the skilled artisan will appreciate that notwithstanding that a sequence, for example a primer, may have a portion that is complementary to a sense strand or sequence of a target sequence, and may also have a portion that is complementary to an antisense strand or sequence of the same target sequence, the target sequence is not required to be double stranded, and may, in fact, be single-stranded. In other words, every single-stranded nucleic acid in this regard can be considered to have a sense sequence (e.g. the strand's sequence itself) and an antisense sequence (which may for example be purely hypothetical in one respect, but which exists at least transiently for example during some portion of an amplification reaction). The skilled artisan will also understand that the foregoing does not preclude a double stranded target nucleic acid sequence, wherein both the sense and antisense sequence exist in actuality. In such a case, each of the strands may be considered to be a sense strand or sequence and yet have an antisense sequence (or complementary sequence).

Strand Displacing Polymerases Useful in the Present Invention.

For the amplification of the target nucleic acids in accordance with certain embodiments of the methods provided herein, a DNA polymerase having strand displacement activity is required. Many examples of such polymerases are known in the art. The properties of exemplary strand-displacing polymerases are shown in Table 2.

TABLE 2
DNA polymerases having a strand displacement activity

| | | |
|---|---|---|
| Exo-nucleases | | |
| activity | Strand | Optimal |

| | | 3′-5′ | displacing | temp |
|---|---|---|---|---|
| Polymerase | 5′-3′ | proof | activity | (° C.) |
| Vent DNA | − | ++ | ++ | |
| Vent DNA | − | − | +++ | |
| (exo-) | | | | |
| Deep Vent | − | +++ | ++ | |
| Deep Vent | − | − | ++ | |
| (exo-) | | | | |
| 9° Nm DNA | − | + | +++ | |
| Therminator | − | − | + | 75 |
| Bst DNA Large | − | −− | ++++ | 65 |
| Fragment | | | | |
| Klenow | − | ++ | ++ | 37 |
| Fragment | | | | |
| Klenow | − | − | ++ | 37 |
| Fragment (exo-) | | | | |
| M-MuLV RT | − | − | +++ | 37 |
| Phi 29 | − | ++++ | +++++ | 30 |

Catalytic nucleic acids have been used in combination with in vitro amplification protocols as a means of generating signal thus allowing real time monitoring of amplified nucleic acid target sequences (Todd et al., 2000; U.S. Pat. No. 6,140,055; U.S. Pat. No. 6,201,113; WO 99/45146; PCT/IB99/00848; WO 99/50452). Protocols for determining conditions for concurrent DNAzyme and polymerase activity at high temperature, such as during PCR, have been described (Impey et al., 2000). The skilled artisan will appreciate how the influence of factors, for example DNAzyme arm length, buffer, temperature, divalent ion concentration, and effects of various additives can be determined. DNAzymes are well-suited for use in combination with in vitro amplification strategies since, unlike the majority of protein enzymes, they are not irreversibly denatured by exposure to temperatures typically used during amplification.

DzyNA-PCR is a published strategy for the detection of specific nucleic acid sequences, for example, genetic sequences associated with disease, or with the presence of foreign agents (WO99/45146). The method provides a system that allows homogeneous target sequence amplification coupled with signal amplification and detection in a single closed vessel. The strategy involves amplification of nucleic acid sequences by PCR using a “DzyNA primer” which has target-specific sequence at its 3′ end and the complementary (anti-sense) sequence of a DNAzyme at its 5′ end. DzyNA-PCR protocols that use the 10:23 DNAzyme have been published (Todd et al., 2000). During DzyNA-PCR, amplicons are produced which contain active (sense) copies of DNAzymes and these catalyse the cleavage of a reporter substrate included in the reaction mix. Cleavage of the reporter substrate is indicative of successful amplification of the target nucleic acid sequence. The accumulation of amplicons during DzyNA-PCR can be monitored by changes in fluorescence produced by separation of fluorescent/quencher dye molecules (e.g. FAM/BHQ1 or JOE/BHQ1) incorporated into opposite sides of a DNAzyme cleavage site within a reporter substrate. Real time fluorometric measurements can be performed on the ABI Prism 7700 Sequence Detection System (SDS) or other platforms that allow monitoring of assays in real time. Examples of real time platforms include ABI Prism 7500 SDS, Rotogene 3000, Bio-Rad myQ, Roche Lightcycler 2.0, Stratagene MX 3000p, MJ Research Opticon and the Cepheid SmartCycler.

The ABI PRISM™ 7700 SDS software can be used to monitor the increase in reporter dye fluorescence (e.g. FAM fluorescence at 530 nm) following cleavage of a substrate by DNAzymes amplified during DzyNA-PCR (Todd et al., 2000). The cycle threshold value (Ct) is defined as the cycle when fluorescence exceeds a defined baseline signal (threshold ΔRn) within the log phase of PCR product accumulation (Heid et al., 1996). A calibration curve can be generated when the log of the copy number is plotted against the Ct value. A skilled artisan will appreciate that quantitation of the amount of nucleic acid in reactions can be estimated from the calibration curve. Similarly, the ABI PRISM™ 7700 SDS software can be used to monitor the changes in reporter dye fluorescence following cleavage of the reporter probe by DNA polymerase during TaqMan™ PCR or following hybridization of Molecular Beacons.

### DETAILED DESCRIPTION

In a first of its several aspects, the invention provides methods for detecting the presence of a nucleic acid sequence in a sample. The methods comprise:

(a) providing a primer mixture comprising:
(i) a pair of inner primers; or
(ii) a pair of inner primers and at least one outer primer; or
(iii) a pair of inner primers and at least one loop primer; or
(iv) a pair of inner primers, at least one outer primer, and at least one loop primer.

The pair of inner primers comprises a forward inner primer and a backward inner primer. Each of the inner primers comprises a first portion that hybridizes to a sense sequence of a target nucleic acid sequence, and a second portion that hybridizes to an antisense sequence (or complement) of the target nucleic acid sequence. The outer primers, where present, are complementary to, or hybridize with a portion of the target nucleic acid sequence.

The loop primers, where present, each comprise a portion complementary to a single-stranded loop region on an amplicon produced from the extension of the forward inner primer or the backward inner primer.

At least one primer in the primer mixture comprises an antisense sequence of a catalytic nucleic acid. The antisense sequence is located or positioned such that a corresponding sense strand of said catalytic nucleic acid is incorporated into an amplicon produced during the amplification.

More specifically, when the primer mixture does not comprise any loop primers, the antisense sequence of the catalytic nucleic acid is positioned between the first and the second portion of one or both the forward or backward inner primer. However, when the primer mixture comprises at least one loop primer, the antisense sequence of a catalytic nucleic acid can be positioned two different ways. The antisense sequence of the catalytic nucleic acid is located between the first and the second portions of one or both of the inner primers (i.e., either the forward or backward inner primer, or both, as above where no loop primer is present), or it is positioned at the 5′ end of one or more loop primers, or the antisense sequence of the catalytic nucleic acid is located in both of the foregoing locations.

(b) contacting the sample with the primer mixture under conditions permitting catalytic nucleic acid activity and target-dependent, primer-initiated, DNA polymerase-mediated nucleic acid amplification. The DNA polymerase has strand displacement activity.

(c) incubating the sample with the primer mixture to allow the primer mixture to initiate amplification, when (and only when) the target nucleic acid sequence is present, to produce amplicons comprising the catalytic nucleic acid; and

(d) determining the presence of the catalytic nucleic acid activity, thereby determining the presence of target nucleic acid sequence in the sample.

In various preferred embodiments, the primer mixture comprises
(i) a pair of inner primers, but no loop or outer primers; or
(ii) a pair of inner primers and at least one outer primer, but no loop primers; or
(iii) a pair of inner primers and at least one loop primer, but no outer primers; or
(iv) a pair of inner primers, at least one outer primer, and at least one loop primer.

Where it is stated that the primer mixture can comprise “at least one” outer primer or “at least one” loop primer, it is to be understood that the methods can be practiced with neither outer nor loop primers, and thus it is possible to practice with only one of either or both outer or loop primers present for a particular target nucleic acid sequence. More preferably, outer and loop primers are used in pairs. In some cases, three or more such primers can be used in a particular embodiment of the methods provided herein. The working examples include reactions that comprise at least three loop primers. Preferably, if there are more than one pair of loop primers present for a particular target nucleic acid sequence, one or more of those loop primers will include the antisense sequence of a catalytic nucleic acid at the 5′ end.

The skilled artisan will appreciate that with respect to the primers, it is sometimes herein stated that the primer hybridizes with the target nucleic acid sequence or hybridizes with a sequence that is the antisense sequence of the target nucleic acid molecule, in this context, the hybridization will occur under the conditions required for amplification and detection, but may not necessarily occur under other conditions, for example, conditions that support neither amplification nor detection of the target sequence, or only one of the two.

The use of the terms “inner primer”, “outer primer”, or “loop primer” herein is consistent with the terminology of the published LAMP amplification procedures, and commercial kits therefor. It should be noted that in many embodiments herein, the amplification procedures are varied from the standard LAMP amplification procedure. As is described in the Definitions section hereinabove, LAMP procedures require 4 primers to recognize six sequences or portions related to the target nucleic acid molecule. Accelerated LAMP procedure require 6 primers to accomplish the same. The skilled artisan will appreciate that it has been established herein that successful amplification and detection of target nucleic acid sequences with catalytic nucleic acid enzymes can be practiced with the inclusion of a primer mixture comprising only a pair of inner primers, wherein there are two primers recognizing four sequences related to the target nucleic acid molecule. The methods provided herein also permit, in various embodiments, the inclusion of a pair of inner primers with at least one loop primer but no outer primers as described above.

The contacting step may be performed in any type of reaction vessel or any chamber sufficient to hold the sample and the reaction components, including those providing the conditions for target-dependent, primer-initiated, DNA polymerase-mediated nucleic acid amplification of the target sequence and catalytic nucleic acid activity. The skilled artisan will appreciate that such conditions include the required buffers, ions, enzymes, precursors, co-factors, components, as well as appropriate temperatures (or temperature cycles where cycling is used) and the like. The methods are target-dependent in that amplification of the target sequence only occurs in the presence of the specific target sequence.

To the extent the methods are DNA-polymerase mediated, any DNA polymerase with the ability to extend the primers used herein will suffice. Of particular interest are those DNA polymerases that have strand displacement activity. DNA polymerases with such activity are known in the art and an exemplary list is provided herein above in Table 2.

The incubation step may be conducted at any temperature permissive of both amplification and catalytic nucleic acid activity. Presently preferred methods are isothermal, or at least substantially isothermal, wherein at least the contacting and incubating steps are conducted at the same temperature. In one embodiment, the incubation step is conducted at temperature less than about 62° C., but above ambient temperature. Preferred incubation steps are conducted at a temperature of about 37° C. to about 58° C. Also preferred are methods wherein the incubation is at a temperature of about 40° C. to about 56° C. Methods wherein the incubation is at any specific temperature from about 50° C. to about 58 or 59° C. are also contemplated for use herein. Incubation temperatures of 45, 46, 47, 48, 49 and 50° C. are also contemplated for use herein.

With respect to the location of the antisense strand of the catalytic nucleic acid within a primer, the skilled artisan will appreciate that there are numerous possible choices for placing such a sequence. The inventors have found that there are only a few such locations that are actually beneficial in the various embodiments provided herein. As discussed above, at least one primer in the primer mixture must comprise such an antisense sequence of a catalytic nucleic acid located or positioned such that a corresponding sense strand, or active strand, of said catalytic nucleic acid is incorporated into at least one amplicon produced during the amplification of the target nucleic acid molecule. This is a novel aspect of the method. When there are no loop primers present in the primer mixture, the antisense sequence of the catalytic nucleic acid is positioned between the first and the second portion of one or both the forward or backward inner primer. This would be the case wherever the primer mixture comprises only inner primers, and also where the primer mixture comprises only inner and one or more outer primers. The skilled artisan will appreciate that for detection, the antisense sequence cannot be located or positioned solely within, or on, one or more of the outer primers, since such a configuration will not allow the antisense sequence to be incorporated into an appropriate amplicon.

When at least one loop primer is present in the primer mixture, the antisense sequence of the catalytic nucleic acid can be positioned in two ways. The antisense sequence of the catalytic nucleic acid is located between the first and the second portions of one or both of the inner primers (i.e., the forward and/or backward inner primer), or it is positioned on one or more loop primers at the 5′ end. In one embodiment, the antisense sequence of the catalytic nucleic acid is located in both of the foregoing locations for a given reaction.

In the basic method provided above, the presence or absence of the target nucleic acid molecule is determined based on the presence or absence of the catalytic nucleic acid activity. In this embodiment the method provides a simple Yes/No result for the presence of the target molecule in the sample. The skilled artisan will appreciate that there are an infinite number of useful applications of such a test.

In another embodiment, the method comprises the further step of determining the amount of catalytic nucleic acid activity. Such an embodiment is particularly useful where a relative amount of a target nucleic acid is of interest. While not completely quantitative, it is possible to compare the relative amount of a target present in a number of reactions conducted under the same conditions.

In yet another embodiment, the method further comprises the step of comparing the amount of activity so determined to a known standard. The skilled artisan will appreciate that once determined, such a known standard allows the quantitative determination of the specific amount of the target nucleic acid sequence present in the sample. In a preferred embodiment a standard curve is constructed from a plurality of known standards.

In certain embodiments, the catalytic nucleic acid is a DNAzyme. Any DNAzyme can be used in accordance with the methods provided herein. Presently preferred DNAzymes include, but are not limited to, 10:23 DNAzymes, and 8:17 DNAzymes.

In other embodiments, the catalytic nucleic acid is a ribozyme. Any ribozyme can be used in accordance with the methods provided herein. One type of ribozyme suitable for use herein is the hammerhead ribozyme. When the antisense sequence of a ribozyme is included in a primer for a particular target nucleic acid sequence such that amplicons comprising a ribozyme are to be produced corresponding to that target sequence, an RNA polymerase and an appropriate promoter are also required in the reaction system or assay.

The target nucleic acid sequence is DNA in certain embodiments, and RNA in other embodiments. Where the sample comprises RNA to be detected, the method further comprises the step of reverse transcribing the sample prior to the contacting step (c).

The methods plainly comprise the use of an active catalytic nucleic acid for detection herein, such as for amplification of a detectable signal. In one preferred embodiment, the catalytic nucleic acid activity comprises the modification of a detectable chemical substrate. The modification preferably comprises formation or cleavage of one or more phosphodiester bonds, or ligation or cleavage of at least one nucleic acid. In certain embodiments that are exemplified herein, the detectable chemical substrate is a fluorescently-labeled nucleic acid molecule, and the modification is cleavage thereof. The fluorescently-labeled nucleic acid molecule is a DNA/RNA chimera in one embodiment that is presently preferred.

Any naturally-occurring or nonnaturally-occurring nucleic acid that is suited or adaptable for amplification can be a target nucleic acid sequence for use herein. The target nucleic acid sequence is from a human, a bacterium, a mycoplasma, an archaea, a plant, an animal, or a virus in certain embodiments. In various embodiments, the target nucleic acid sequence is from a human. The methods are particularly useful as diagnostic tools in assessing human health, and e.g. disease conditions. The methods are also applicable to a variety of other diagnostic applications. For example, such methods may be useful in testing for suspected accidental or intentional release of any of a broad array of different etiological agents. The presence of the target nucleic acid sequence in the sample is indicative of a genetic disorder in one embodiment, in another embodiment, the absence of a target is an indication of such a disorder.

Samples for use in the methods provided herein may be derived from any source, and the methods provided are particularly well-suited for samples which are clinical, forensic, environmental, agricultural, or veterinary in terms of their origin or source. Such broad categories are not mutually exclusive as the skilled artisan will recognize, for example a sample taken from a farm where animals are raised may be deemed environmental, agricultural, or veterinary depending on the circumstances. The disclosure of certain of such sources is not to the exclusion of others for use herein, but rather is to help inform as to examples of the samples suitable for use with the instant methods.

In one embodiment, the primer-initiated nucleic acid amplification is LAMP per se, in another embodiment it is not a standard LAMP procedure, for example, in its selection and use of inner primers, and outer and/or loop primers. In particular embodiments, the primer mixture comprises at least a pair of inner primers but no outer primers or loop primers. In other embodiments the primer mixture comprises at least a pair of inner primers and at least one outer primer, but no loop primers. In other embodiments the primer mixture comprises a pair of inner primers and at least one loop primer, but no outer primers. In yet other embodiments the primer mixture comprises each of a pair of inner primers, at least one outer primer, and at least one loop primer.

In another of its several aspects, the invention provides methods for detecting the presence of each of a plurality of target nucleic acid sequences in a sample, the methods generally comprise:

(a) providing a primer mixture comprising, for each of the plurality of target nucleic acid sequences to be detected at least:
(i) a pair of inner primers; or
(ii) a pair of inner primers and at least one outer primer; or
(iii) a pair of inner primers and at least one loop primer; or
(iv) a pair of inner primers, at least one outer primer, and at least one loop primer;

(b) contacting the sample with the primer mixture under conditions permitting catalytic nucleic acid activity and target sequence-dependent, primer-initiated, DNA polymerase-mediated nucleic acid amplification;

wherein the DNA polymerase has strand displacement activity;

(c) incubating the sample with the primer mixture to allow the primer mixture to initiate amplification of each of the plurality of target nucleic acid sequences, when that target nucleic acid sequence is present, to produce amplicons comprising the distinctly detectable catalytic nucleic acid; and

(d) determining the presence of each of the uniquely detectable catalytic nucleic acid activities, thereby determining the presence of the corresponding target nucleic acid sequence in the sample;

wherein:

Each pair of inner primers comprises a forward inner primer and a backward inner primer. Each inner primer comprises a first portion that hybridizes to a sense sequence of at least one of the plurality of target nucleic acid sequences, and a second portion that hybridizes to an antisense sequence (or complement) of that target nucleic acid sequence.

Each outer primer, where present, hybridizes to a portion of at least one of the plurality of target nucleic acid sequences.

Each loop primer, where present, comprises a portion complementary to a single stranded loop region on an amplicon produced from the extension of at least one forward inner primer or backward inner primer corresponding to at least one of the plurality of target nucleic acid sequences.

For each one of the plurality of target nucleic acid sequences, at least one primer in the primer mixture comprises an antisense sequence of a distinctly detectable catalytic nucleic acid such that a corresponding sense strand of said distinctly detectable catalytic nucleic acid is incorporated in an amplicon produced during amplification of that target nucleic acid sequence.

For each of the plurality of target nucleic acid sequences, when the primer mixture does not comprise any loop primers for that particular target nucleic acid sequence, the antisense sequence of a distinctly detectable catalytic nucleic acid is positioned between the first and the second portions of one or both of the forward or backward inner primers. And, for each of the plurality of target nucleic acid sequences, when the primer mixture does comprise at least one loop primer for that target nucleic acid sequence, the antisense sequence of a distinctly detectable catalytic nucleic acid is positioned between the first and the second portions of one or both of the forward or backward inner primers, or at the 5′ end of at least one of the loop primers, or even in both of the foregoing locations (i.e. inner and loop primer positions).

The skilled artisan will appreciate that this aspect of the invention provides a means for amplifying and detecting each of a plurality of target nucleic acids in a sample. The number of the plurality of such targets may be 2 or more. The plurality may comprise a very large number of sequences which are closely related, or the sequences may be unrelated. Such assays have numerous applications that need not be specified here as the skilled artisan will understand their use. It is also to be understood that this aspect of the invention shares many embodiments and alternatives that are provided in the first aspect of the invention, although the methods provided therein are directed to methods for detecting a single target nucleic acid in a sample, whether in a background of other sequences, e.g. a plurality of sequences not being detected, or whether in relative or complete isolation.

Thus, as above, the primer mixture comprises in various embodiments, for each of the plurality of target nucleic acid sequences
(i) a pair of inner primers, but no loop or outer primers; or
(ii) a pair of inner primers and at least one outer primer, but no loop primers; or
(iii) a pair of inner primers and at least one loop primer, but no outer primers; or
(iv) a pair of inner primers, at least one outer primer, and at least one loop primer.

It is to be appreciated that the above discussion of the primer mixture pertains to the primers provided therein for each of the plurality of target nucleic acid sequences. It is contemplated that in various applications of the methods, the primer mixture may contain, for example primers (i) above for one target sequence, primers (ii) for a another target sequence, and so on. There is nothing that precludes the use of any combination of primers (i), (ii), (iii), and/or (iv) above when preparing and providing a primer mixture in accordance with any of the methods disclosed herein. In other words the primer mixture need not comprise the exact same types of primers (inner, outer and loop) for each of a plurality of target nucleic acid sequences. This is particularly useful in those methods wherein detection is of each of, or any of, a plurality of sequences.

Here too, statements that the primer hybridizes with the target nucleic acid sequence or hybridizes with a sequence that is the antisense sequence of the target nucleic acid molecule mean only that the hybridization will occur under the conditions required for amplification and detection; it may not necessarily occur under other conditions. Also as set forth in the definitions the use of the terms sense and antisense with respect to the target nucleic acid sequences is not an indication that the target molecules are double-stranded.

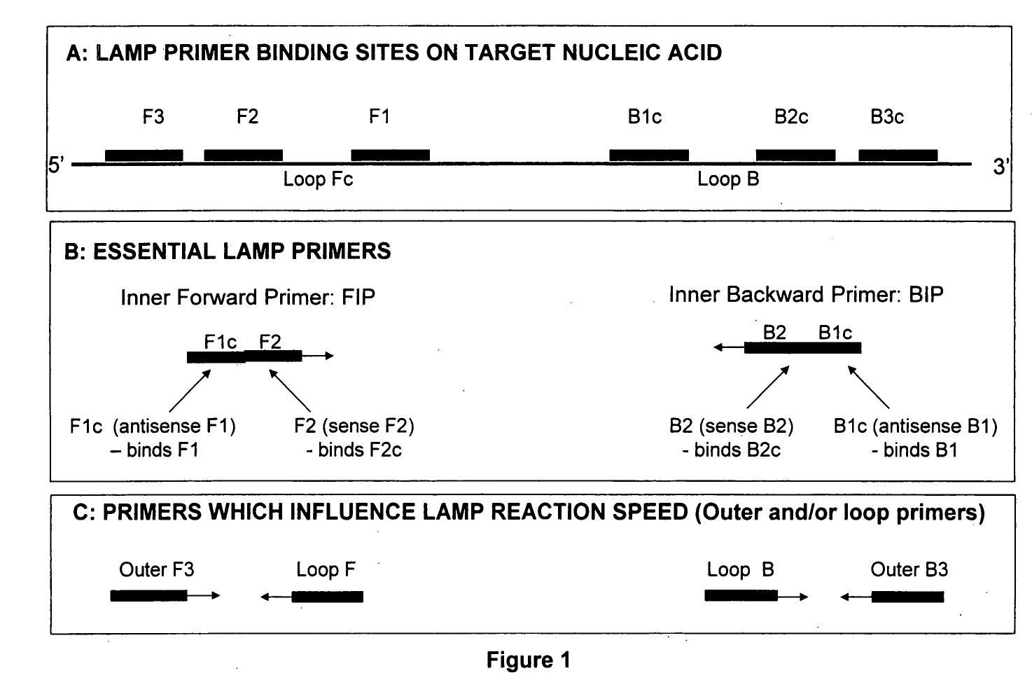

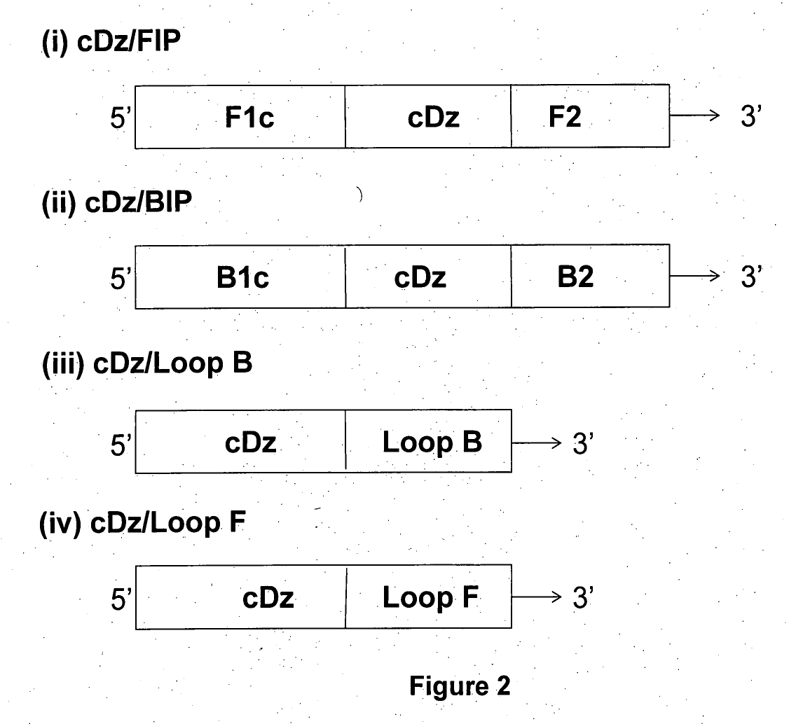

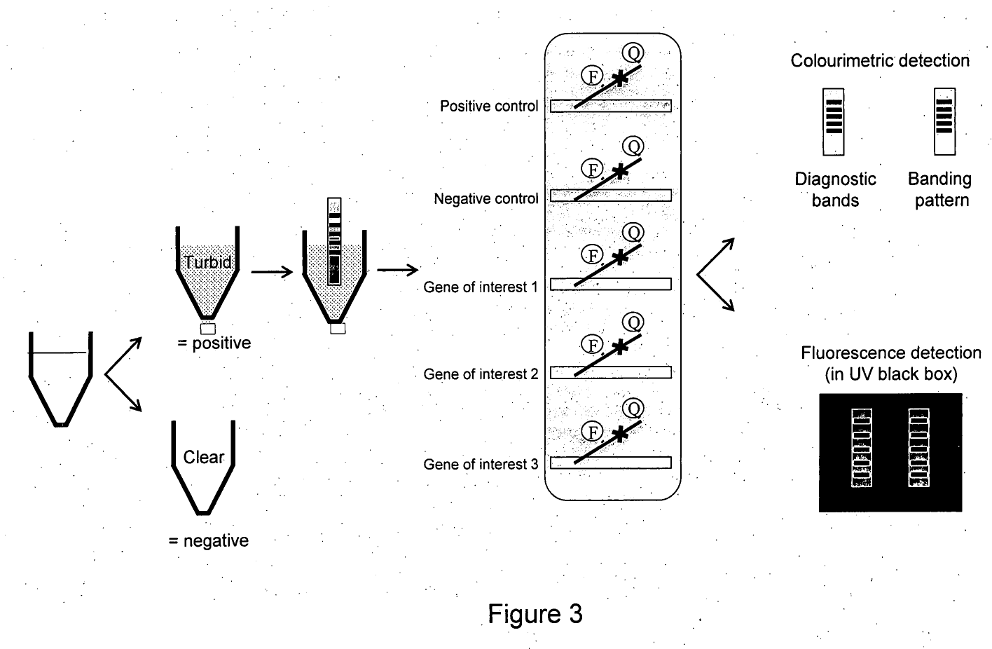

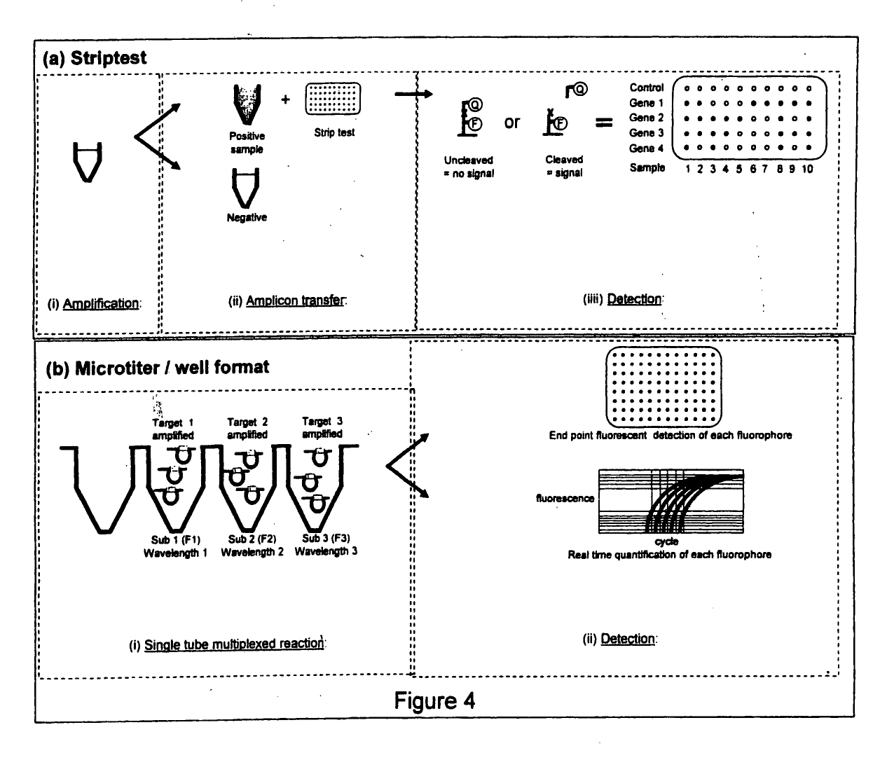

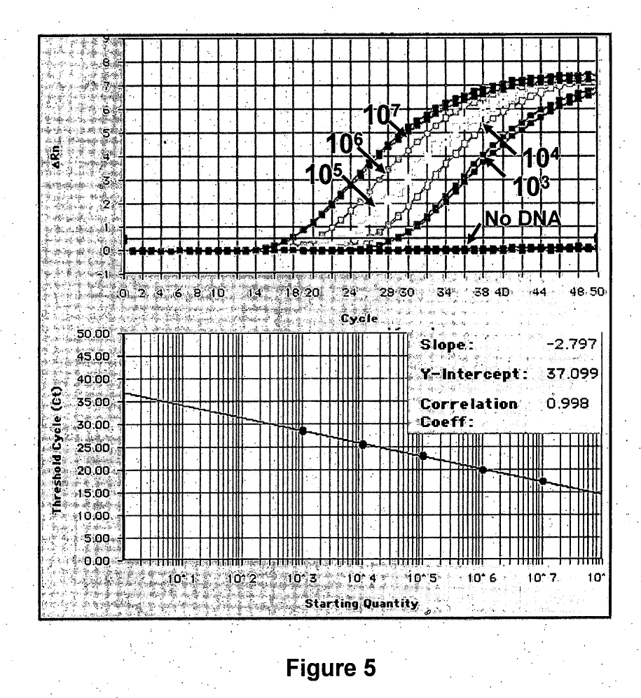

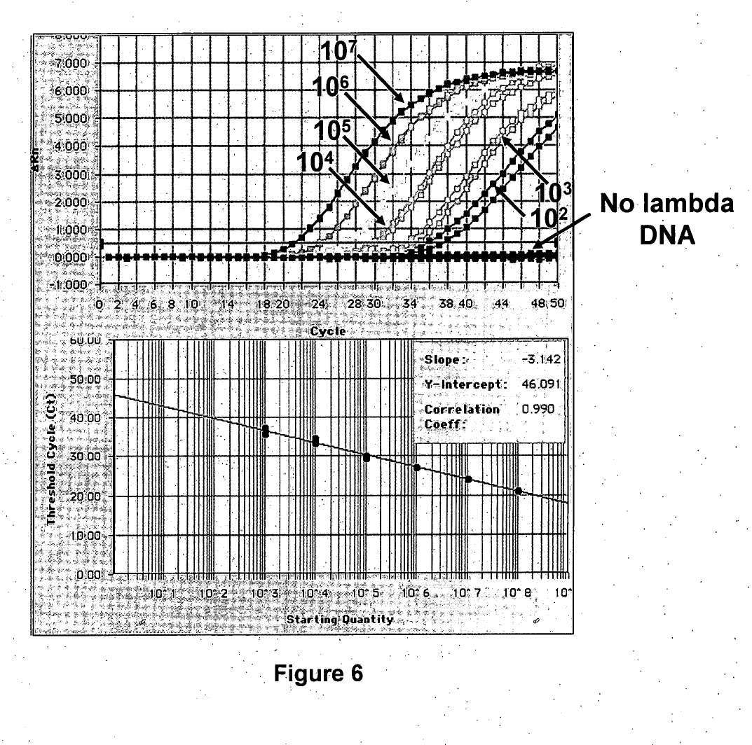

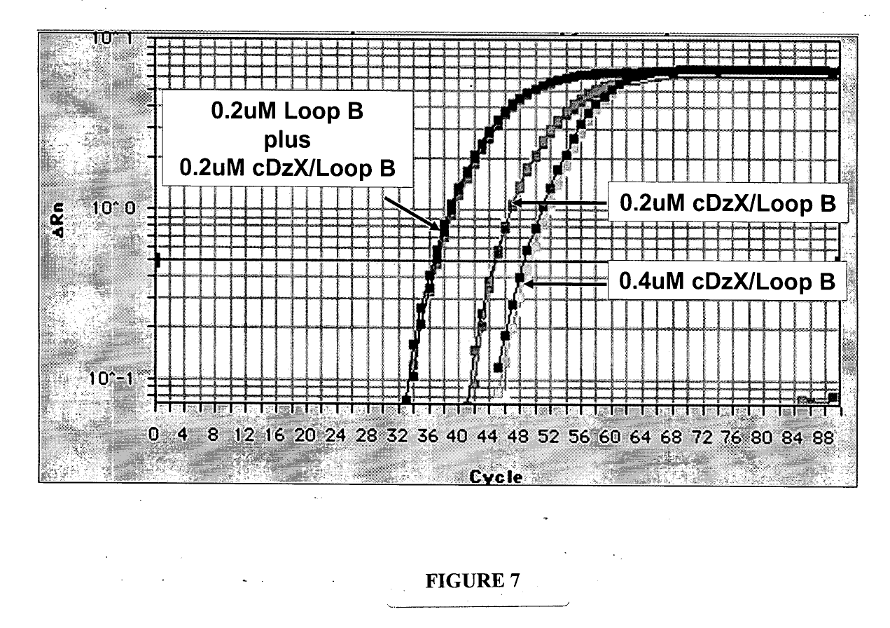

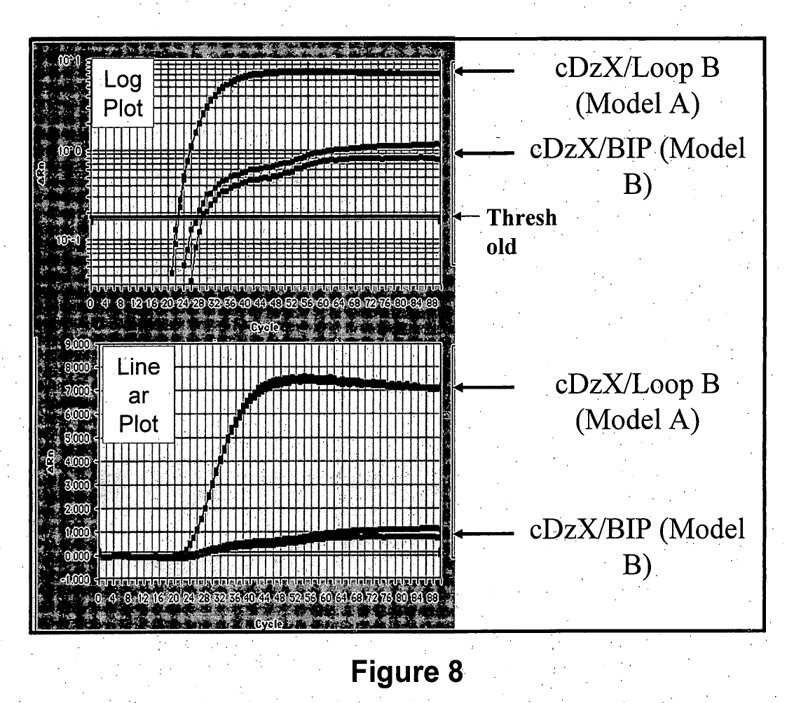

The use of the terms “inner primer”, “outer primers”, or “loop primers” here, like above is consistent with the terminology developed in LAMP amplification literature, however, the actual amplification procedures employed may be neither a standard nor accelerated LAMP amplification (“nonstandard LAMP”), for example in that the requirement for outer primers in LAMP is relaxed herein. FIG. 1 depicts such primers in relation to a target nucleic acid sequence.

The step of contacting the sample with the primer mixture may be performed in any type of reaction vessel or any chamber sufficient to hold the sample and the reaction components, including those providing the conditions for target-dependent, primer-initiated, DNA polymerase-mediated nucleic acid amplification of the target sequence and catalytic nucleic acid activity. Presently preferred are tubes, particularly microtubes, and wells, e.g. microwells. The skilled artisan will appreciate that the conditions for target-dependent, primer-initiated, DNA polymerase-mediated nucleic acid amplification include everything but the target itself—as that is the analyte the presence of which is to be determined. Thus conditions include the required buffers, ions, enzymes, precursors, co-factors, components, as well as appropriate temperatures (or temperature cycles where cycling is used) and the like. The methods are target-dependent in that amplification of the target sequence only occurs in the presence of the specific target sequence.

To the extent the methods are DNA-polymerase mediated, any DNA polymerase with the ability to extend the primers used herein will suffice. Of particular interest are those DNA polymerases that have strand displacement activity. DNA polymerases with such activity are known in the art and an exemplary list is provided herein above in Table 2.

The incubation step may be conducted at any temperature permissive of both amplification and catalytic nucleic acid activity. Although in certain embodiments, temperature cycling is compatible with combined amplification and detection reactions, presently preferred methods are isothermal, or at least substantially isothermal, wherein at least the contacting and incubating steps are conducted at the same temperature.

In one embodiment, the incubation step is conducted at temperature less than about 62° C., but above ambient temperature. Other preferred incubation steps are conducted at a temperature of about 37° C. to about 58° C. Also preferred are methods wherein the incubation is at a temperature of about 40° C. to about 56° C. Methods wherein the incubation is at any specific temperature from about 50° C. to about 58 or 59° C. are also contemplated for use herein. Incubation temperatures of 45, 46, 47, 48, 49 and 50° C. are also contemplated for use herein.

With respect to the location of the antisense strand of the catalytic nucleic acid within a primer, the skilled artisan will appreciate that there are numerous possible choices for placing such a sequence. The inventors have found that there are only a few such locations that are actually beneficial in the various embodiments provided herein. For each of the plurality of targets, at least one primer in the primer mixture must comprise such an antisense sequence of a catalytic nucleic acid located or positioned such that a corresponding sense strand, or active strand, of said catalytic nucleic acid is incorporated into at least one amplicon produced during the amplification of that particular target nucleic acid molecule. The activity of the catalytic nucleic acid corresponding to each of the plurality of target nucleic acid sequences to be detected must be distinctly detectable to allow the individual analytes (target nucleic acid sequences) to be detected. When there are no loop primers present in the primer mixture for a particular target nucleic acid sequence of the plurality, the antisense sequence of the catalytic nucleic acid corresponding to that target nucleic acid sequence is positioned between the first and the second portion of one or both the forward or backward inner primer for that target sequence. This is the case wherever the primer mixture comprises only inner primers, and also where the primer mixture comprises only inner and outer primers. The skilled artisan will appreciate, that as in the previous aspect pf the invention described above, the antisense sequence cannot be located or positioned solely within, or on, one or more outer primers for any target nucleic acid sequence, since such a configuration will not allow the antisense sequence to be incorporated into an appropriate amplicon such that the activity could be detected.

When at least one loop primer for a given target sequence are present in the primer mixture, the antisense sequence of the catalytic nucleic acid corresponding to that target can be positioned in two ways. The antisense sequence of the catalytic nucleic acid is located between the first and the second portions of one or both of the inner primers (i.e., the forward and/or backward inner primer) for that target sequence, or it is positioned on one or more loop primers for that sequence, at the 5′ end of the respective loop primer. In one embodiment, the antisense sequence of the catalytic nucleic acid is located in both of the foregoing locations for a given target.

In the multiplexed method provided above, the presence or absence of each of the plurality of target nucleic acid sequences is determined based on the presence or absence of the distinctly detectable catalytic nucleic acid activity corresponding to that target. In this embodiment the method provides a simple Yes/No result for the presence of each particular target molecule in the sample. One target sequence of the plurality being tested for may be present in the sample while another may be absent from the sample. The skilled artisan will appreciate that there are an infinite number of useful applications of such tests.

In another embodiment, the method comprises the further step of determining the amount of at least one of the distinctly detectable catalytic nucleic acid activities. Such an embodiment is particularly useful where a relative amount of one or more of the plurality of target nucleic acid sequences is of interest. While not completely quantitative, it is possible to compare the relative amount of each of the plurality of target nucleic acids present, or a subgroup thereof, in a number of reactions conducted under the same conditions, for example, in parallel assays.

In yet another embodiment, the method further comprises the step of comparing the amount of each activity so determined to a known standard for that activity. The skilled artisan will appreciate that once determined, such a known standard allows the quantitative determination of the specific amount in the sample of the particular target nucleic acid sequence corresponding to that standard. In a preferred embodiment a standard curve is constructed from a plurality of known standards for each sample to be quantified.

In certain embodiments, one or more of the catalytic nucleic acids with distinctly detectable activity is a DNAzyme. Any DNAzyme can be used in accordance with the methods provided herein. Presently preferred DNAzymes include, but are not limited to, 10:23 DNAzymes, and 8:17 DNAzymes.

In other embodiments, at least one of the distinctly detectable catalytic nucleic acid activities is a ribozyme. Any ribozyme can be used in accordance with the methods provided herein. One type of ribozyme suitable for use herein is the hammerhead ribozyme. The skilled artisan will understand that an RNA polymerase and an sufficient promoter sequence for its function are also required in the reaction system or assay (and thus are deemed present in the conditions permitting both amplification and catalytic nucleic acid activity (i.e. detection) when the antisense sequence of a ribozyme is included in a primer for a particular target nucleic acid sequence such that amplicons comprising a ribozyme are to be produced corresponding to that target sequence.

At least one of the plurality of target nucleic acid sequences is DNA in certain embodiments, and RNA in other embodiments. In one embodiment the target sequences are all DNA, in another they are all RNA. Where the sample comprises RNA to be detected, the method further comprises the step of reverse transcribing the sample prior to the contacting step (c).

The methods provided in this aspect plainly comprise the use of active catalytic nucleic acids for detection herein, such as for amplification of a detectable signal. In one preferred embodiment, each catalytic nucleic acid activity comprises the modification of a chemical substrate, the modification of which is distinctly detectable. The modification preferably comprises formation or cleavage of one or more phosphodiester bonds, or ligation or cleavage of at least one nucleic acid. In certain embodiments that are exemplified herein, the detectable chemical substrate is a fluorescently-labeled nucleic acid molecule, and the modification is cleavage thereof. In a preferred embodiment each catalytic nucleic acid has a corresponding substrate with a different fluorescent label. The fluorescently-labeled nucleic acid molecule is a DNA/RNA chimera in one embodiment that is presently preferred. In another embodiment the modification, for example cleavage of the substrate, produces a signal that can be monitored in real time on a device adapted for reading such signals.

Any naturally-occurring or nonnaturally-occurring nucleic acid that is suited or adaptable for amplification can be one of the plurality of target nucleic acid sequences for use herein. The plurality of target sequences need not all be from the same organism. At least one of the plurality of target nucleic acid sequences preferably is from a human, a bacterium, a mycoplasma, an archaea, a plant, an animal, or a virus, in certain embodiments. In various embodiments, at least one of the target nucleic acid sequences is from a human. The methods are particularly useful as diagnostic tools in assessing human health, and e.g. disease conditions. They are also applicable to a variety of other diagnostic applications. For example, such methods may be applicable in testing for suspected accidental or intentional release of a broad array of different etiological agents. In one embodiment, the presence of at least one of the plurality of target nucleic acid sequences in the sample is indicative of a genetic disorder in a person or animal from whom the sample originates, in another embodiment, the absence of any such target sequences, or of a particular such target sequence is an indication of such a disorder.

Samples for use in the methods provided herein in this aspect of the invention may be derived from any source, and the methods provided are particularly well-suited for samples which are clinical, forensic, environmental, agricultural, or veterinary in terms of their origin or source. Such broad categories are not mutually exclusive, for example, a sample may be deemed environmental, agricultural, and veterinary depending on the circumstances and its source. The disclosure of certain of such sources is not to the exclusion of others for use herein, but rather exemplifies the types of samples suitable for use with the instant methods.

In one embodiment, the primer-initiated nucleic acid amplification is LAMP, in another embodiment it is a nonstandard LAMP amplification, for example in its selection and use of inner, outer, and/or loop primers. In particular embodiments, the primer mixture comprises at least a pair of inner primers but no outer primers or loop primers for at least one of the plurality of target nucleic acid sequences. In other embodiments the primer mixture comprises for at least one of the plurality of target nucleic acid sequences at least a pair of inner primers and at least one outer primer, but no loop primers for that sequence. In other embodiments the primer mixture comprises for at least one of the plurality of target nucleic acid sequences a pair of inner primers and at least one loop primer, but no outer primers for that sequence. In yet other embodiments the primer mixture comprises, for at least one of the plurality of target nucleic acid sequences, a pair of inner primers, at least one outer primer, and at least one loop primer. As discussed above, in a given assay, there is nothing that precludes a combination of the foregoing for the plurality of target nucleic acid sequences to be detected. Thus the primers for one target, e.g. a relatively abundant target sequence, may include only inner primers, while the primers for a different target, e.g. a less abundant target, may include inner, outer, and loop primers. Alternatively each of the plurality of target nucleic acid sequences may have analogous sets of primers (e.g. only inner primers for each target).

In a presently preferred embodiment, for each target the primer mixture comprises a pair of inner primers, at least one outer primer, and at least one loop primer. In one embodiment, the primers include various modifications that have been found useful herein, for example a backbone modification. Modifications to the nucleic acid backbone can be made including, but not limited to, the inclusion of blocking moieties to prevent polymerase mediated chain extension of the primer on the template. Such blocking moieties include, but are not limited, to hexethylene glycol (HEG) monomers or 2-O-alkyl RNA. Such modifications are contemplated for use in any of the aspects of the instant invention. For example, primers for either the single target methods or those methods for detecting each of or any of a plurality of nucleic acids may comprise such modifications. In a preferred embodiment, one or more of the primers comprise a HEG modification to their backbone, preferably the backbone modifications are to one or more primers comprising the antisense sequence of the catalytic nucleic acid. In particular, examples of the use of backbone modifications with HEG are provided in the working examples provided herewith to exemplify various aspects of the invention.

In another its several aspects, the invention provides methods of detecting the presence of any of a plurality of target nucleic acid sequences in a sample. The methods comprise the steps of:

(a) providing a primer mixture comprising one or more primers sufficient for amplifying each of the plurality of target nucleic acid sequences to be detected;

wherein for each of the plurality of target nucleic acid sequences, there is at least one primer in said primer mixture comprising an antisense sequence of a catalytic nucleic acid such that a corresponding sense strand of said catalytic nucleic acid is incorporated into an amplicon produced during amplification of that target nucleic acid sequence;

(b) contacting the sample with the primer mixture under conditions permitting catalytic nucleic acid activity and target sequence-dependent, primer-initiated, DNA polymerase-mediated nucleic acid amplification;

(c) incubating the sample with the primer mixture to allow the primer mixture to initiate amplification of any of the plurality of target nucleic acid sequences, when that target nucleic acid sequence is present, to produce amplicons comprising the catalytic nucleic acid; and

(d) determining the presence of the catalytic nucleic acid activity from an amplicon produced during the amplification of any of the target nucleic acid sequences, thereby determining the presence of any of the target nucleic acid sequences in the sample.

In one embodiment of the foregoing aspect, the amplification can be by any method known that is compatible for the conditions required for catalytic nucleic acid activity. Particularly preferred methods include PCR, SDA, RCA, LAMP, TMA, 3SR, or NASBA.

In one embodiment of the method the DNA polymerase has strand displacement activity, and the primer mixture comprises at least:
(i) a pair of inner primers; or
(ii) a pair of inner primers and at least one outer primer; or
(iii) a pair of inner primers and at least one loop primer; or
(iv) a pair of inner primers, at least one outer primer, and at least one loop primer;

wherein:

Each pair of inner primers comprises a forward inner primer and a backward inner primer. Each of the inner primers comprises a first portion that hybridizes to a sense sequence of at least one of the plurality of target nucleic acid sequences, and a second portion that hybridizes to an antisense sequence of that target nucleic acid sequence.

Each outer primer, where present, hybridizes to a portion of at least one of the plurality of target nucleic acid sequences.

Each of the loop primers, where present, comprise a portion complementary to a single stranded loop region on an amplicon produced from the extension of at least one forward inner primer or backward inner primer corresponding to at least one of the plurality of target nucleic acid sequences.

For each of the plurality of target nucleic acid sequences, when the primer mixture does not comprise any loop primers for that target nucleic acid sequence, the antisense sequence of a catalytic nucleic acid is positioned between the first and the second portion of one or both of the forward and backward inner primers. And, for each of the plurality of target nucleic acid sequences, when the primer mixture comprises at least one loop primer for that target nucleic acid sequence, the antisense sequence of a catalytic nucleic acid is positioned between the first and the second portion of one or both of the forward or backward inner primer, or at the 5′ end of one or more loop primers, or at both such locations.

As with the prior aspects of the invention, there is nothing that precludes the use of a different set of primers from the groups of (i), (ii), (iii), and (iv) for each of the plurality of targets, and any given primer mixture can comprise any combination of primer sets (i), (ii), (iii), and/or (iv) above, without limitation so long as the base requirements are satisfied and that each of the plurality of target nucleic acid sequences can be amplified if present in the sample, and detected when amplified.

As with the other aspects of the invention, statements that the primer hybridizes with the target nucleic acid sequence or hybridizes with a sequence that is the antisense sequence of the target nucleic acid molecule mean only that the hybridization will occur under the conditions required for amplification and detection, but may not necessarily occur under other conditions. Also as set forth in the definitions the use of the terms sense and antisense with respect to the target nucleic acid sequences is not an indication that the target molecules are double-stranded.

The use of the terms “inner primers”, “outer primers”, or “loop primers” here, like above is consistent with the terminology developed in LAMP amplification literature, however, the actual amplification procedures employed may not be a standard LAMP protocol, i.e. they may be nonstandard LAMP amplification in that the requirement for outer primers in LAMP is relaxed herein. FIG. 1 depicts such primers in relation to a target nucleic acid sequence.

The step of contacting the sample with the primer mixture may be performed in any type of reaction vessel or any chamber sufficient to hold the sample and the reaction components, including those providing the conditions for target-dependent, primer-initiated, DNA polymerase-mediated nucleic acid amplification of the target sequence and catalytic nucleic acid activity. Presently preferred are tubes, particularly microtubes, and wells, e.g. microwells. The conditions for target-dependent, primer-initiated, DNA polymerase-mediated nucleic acid amplification include everything but the target sequence to be determined. Thus conditions include the required buffers, ions, enzymes, precursors, co-factors, components, as well as appropriate temperatures (or temperature cycles where cycling is used) and the like. The methods are target-dependent in that amplification of the target sequence only occurs in the presence of the specific target sequence, however in this aspect of the invention, the presence of any one of the plurality of the target nucleic acid sequences is sufficient to provide a positive result for the test or assay in the YES/NO embodiment described below.

To the extent the methods are DNA-polymerase mediated, any DNA polymerase with the ability to extend the primers used herein will suffice. DNA polymerases without strand displacement activity are preferred in several of the amplification methods useful with this aspect of the invention. Some of these DNA polymerases require high temperatures for optimal activity, and thus temperature cycling may be preferred for such embodiments. Also of interest for use herein are those DNA polymerases that do have strand displacement activity. DNA polymerases with such activity are known in the art and an exemplary list is provided herein above in Table 2.

The incubation step may be conducted at any temperature permissive of both amplification and catalytic nucleic acid activity. Although in certain embodiments, temperature cycling is compatible with combined amplification and detection reactions, certain presently preferred methods in accordance with this aspect of the invention are isothermal, or at least substantially isothermal, i.e. wherein at least the contacting and incubating steps are conducted at the same temperature. As the skilled artisan would plainly understand, isothermal incubations are not to be selected for those amplification methods requiring thermocycling, such as PCR.

In one embodiment, the incubation step is conducted at constant temperature less than about 62° C., but above ambient temperature. Other preferred incubation steps are conducted at a temperature of about 37° C. to about 58° C. Also preferred are methods wherein the incubation is at a temperature of about 40° C. to about 56° C. Methods wherein the incubation is at any specific temperature from about 50° C. to about 58 or 59° C. are also contemplated for use herein. Incubation temperatures of 45, 46, 47, 48, 49 and 50° C. are also contemplated for use herein.

With respect to the location of the antisense strand of the catalytic nucleic acid within a primer, the skilled artisan will appreciate that there are numerous possible choices for placing such a sequence. The inventors have found that there are only a few such locations that are actually beneficial in the various embodiments provided herein. For each of the plurality of targets, at least one primer in the primer mixture must comprise such an antisense sequence of a catalytic nucleic acid located or positioned such that a corresponding sense strand, or active strand, of said catalytic nucleic acid is incorporated into at least one amplicon produced during the amplification of that particular target nucleic acid molecule. The activity of the catalytic nucleic acid corresponding to each of the plurality of target nucleic acid sequences to be detected need not be distinctly detectable, as the individual analytes (target nucleic acid sequences) are not being detected in these methods.

In one embodiment, when there are no loop primers present in the primer mixture for a particular target nucleic acid sequence of the plurality, the antisense sequence of the catalytic nucleic acid corresponding to that target nucleic acid sequence is positioned between the first and the second portion of one or both the forward or backward inner primer for that target sequence. This is the case wherever the primer mixture comprises only inner primers for a particular target sequence, and also where the primer mixture comprises only inner and outer primers for a particular target sequence. The skilled artisan will appreciate, that as in the previous aspects of the invention employing standard or nonstandard LAMP amplification, the antisense sequence cannot be located or positioned solely within, or on, an outer primer for any target nucleic acid sequence, since such a configuration will not allow the antisense sequence to be incorporated into an appropriate amplicon such that the activity can be detected.

When at least one loop primer for a specific target sequence of the plurality is present in the primer mixture, the antisense sequence of the catalytic nucleic acid corresponding to that target can be positioned two ways. The antisense sequence of the catalytic nucleic acid is located between the first and the second portions of one or both of the inner primers (i.e., the forward and/or backward inner primer) for that target sequence, or it is positioned on one or more of the loop primers for that sequence, at the 5′ end of the respective loop primer. In one embodiment, the antisense sequence of the catalytic nucleic acid is located in both of the foregoing locations for a given target sequence.

In the methods provided in this aspect of the invention, the presence or absence of each of the plurality of target nucleic acid sequences is determined based on the presence or absence of any catalytic nucleic acid activity corresponding to any of the plurality of targets. In this embodiment the method provides a simple Yes/No result for the presence of any of the plurality of target molecules in the sample. One target sequence of the plurality being tested for may be present in the sample while another may be absent from the sample, but the result will be positive in any case. In its simplest form the results are merely YES/NO for the presence of any of a population of target nucleic acids. The skilled artisan will appreciate that there are an infinite number of useful applications of such tests. Broad screening assays are possible using this—for example the plurality of target nucleic acids could comprise a battery of nucleic acids from infectious agents and the test could be employed as an initial or early screen for subjects or patients presenting with symptoms of such infection prior to the administration of a pharmaceutical. Further screening can allow more specific identification and final identification could be accomplished using one of the methods or devices described herein.

In another embodiment, the method comprises the further step of determining the amount of total catalytic nucleic acid activities. Such an embodiment is useful where a relative amount of the plurality of target nucleic acid sequences is of interest. It is possible to compare the relative amount of the plurality of target nucleic acid sequences present, in a number of reactions conducted under the same conditions, for example, in parallel assays.

In yet another embodiment, the method further comprises the step of comparing the amount of each activity so determined to a known standard for the catalytic nucleic acid activity. The skilled artisan will appreciate that once determined, such a known standard allows the quantitative determination of the specific amount of the total target nucleic acid sequence in the sample. In a preferred embodiment a standard curve is constructed from a plurality of known standards to allow the total target nucleic acid in each sample to be quantified.

In certain embodiments, one or more of the catalytic nucleic acids is a DNAzyme. Any DNAzyme, or combination of DNAzymes, can be used in accordance with the methods provided herein. Presently preferred DNAzymes include, but are not limited to, a 10:23 DNAzyme, and a 8:17 DNAzyme.

In other embodiments, at least one of the catalytic nucleic acid activities is a ribozyme. Any ribozyme can be used in accordance with the methods provided herein. One type of ribozyme suitable for use herein is the hammerhead ribozyme. When ribozymes are used for detection, the reaction will comprise an RNA polymerase activity and suitable promoter sequence, as discussed above for other aspects of the invention. In such cases, the amplicons produced will contain an active ribozyme, and the antisense sequence incorporated into a primer will the antisense of a ribozyme.

At least one of the plurality of target nucleic acid sequences is DNA in certain embodiments, and RNA in other embodiments. In one embodiment the target sequences are all DNA, in another they are all RNA. Where the sample comprises RNA to be detected, the method further comprises the step of reverse transcribing the sample prior to the contacting step (c).

The use of active catalytic nucleic acids for detection, such as for amplification of a detectable signal is provided herein. In one preferred embodiment, each catalytic nucleic acid activity comprises the modification of a chemical substrate, the modification of which is detectable. It is not required that such detectable activity be distinct from the detectable activity corresponding to other catalytic nucleic acids, however the use of distinctly detectable catalytic nucleic acids is not incompatible herewith. The substrate modification preferably comprises formation or cleavage of one or more phosphodiester bonds, or ligation or cleavage of at least one nucleic acid. In certain embodiments in the working examples, the detectable chemical substrates are fluorescently-labeled nucleic acid molecules, and the modification is cleavage thereof. In a preferred embodiment each catalytic nucleic acid has a corresponding substrate, however the substrate can be labeled with a universal fluorescent label. In one embodiment a large group of distinct but related nucleic acids can all be detected using a single catalytic enzyme and thus, a universal substrate can be used for detecting any of the plurality of such target nucleic acids.

In one embodiment, the fluorescently-labeled nucleic acid molecule is a DNA/RNA chimera. In another embodiment the modification, for example cleavage of the substrate, produces a signal that can be monitored in real time on a device adapted for reading such signals.

Naturally-occurring and nonnaturally-occurring nucleic acids that are suited for or adaptable for amplification can be among the plurality of target nucleic acid sequences to be detected. The plurality of target sequences need not all be from the same organism. At least one of the plurality of target nucleic acid sequences preferably is from a human, a bacterium, a mycoplasma, an archaea, a plant, an animal, or a virus, in certain embodiments. In various embodiments, at least one of the target nucleic acid sequences is from a human. The methods are particularly useful as diagnostic tools in assessing human health, and e.g. disease conditions. They are also applicable to a variety of other diagnostic applications. For example, such methods may be applicable in testing for suspected accidental or intentional release of any of a broad array of different etiological agents. In one embodiment, the presence of at least one of the plurality of target nucleic acid sequences in the sample is indicative of a genetic disorder in a person or animal from whom the sample originates. In another embodiment, the absence of any such target sequences, or of a particular such target sequence is an indication of such a disorder.

Samples from any source are suitable for use herein. The methods provided are particularly well-suited for samples which are clinical, forensic, environmental, agricultural, or veterinary in terms of their origin or source. Such broad categories are not mutually exclusive here, as has been explained for other aspects of the invention.

In one embodiment, the primer-initiated nucleic acid amplification is LAMP, in another embodiment it is nonstandard LAMP amplification as discussed herein above, for example, in its selection and use of inner primers, and outer and/or loop primers. In particular embodiments, the primer mixture comprises at least a pair of inner primers but no outer primers or loop primers for at least one of the plurality of target nucleic acid sequences. In other embodiments the primer mixture comprises for at least one of the plurality of target nucleic acid sequences at least a pair of inner primers and at least one outer primer, but no loop primers for that sequence. In other embodiments the primer mixture comprises for at least one of the plurality of target nucleic acid sequences a pair of inner primers and at least one loop primer, but no outer primers for that sequence. In yet other embodiments the primer mixture comprises, for at least one of the plurality of target nucleic acid sequences, a pair of inner primers, at least one outer primer, and at least one loop primer. In a given assay, there is nothing that precludes a combination of the foregoing for the plurality of target nucleic acid sequences to be detected. Thus the primers for one target, e.g. a relatively abundant target sequence, may include only inner primers, while the primers for a different target, e.g. a less abundant target, may include inner, outer, and loop primers. Alternatively each of the plurality of target nucleic acid sequences may have analogous sets of primers (e.g. only inner primers for each target).

In a presently preferred embodiment, the primer mixture comprises a pair of inner, outer and loop primers for each of the plurality of target sequences to be detected. One or more of the primers comprise the antisense sequence of the catalytic nucleic acid. In some methods, those primers contain modifications such as the backbone modifications discussed above. In a preferred embodiment, one or more of the primers comprise a HEG modification to their backbone, preferably the backbone modifications are to one or more primers comprising the antisense sequence of the catalytic nucleic acid.

In one embodiment the presence in the sample of any of the target nucleic acid sequences is indicative of a bacterium, a virus, an insect, or a genetically-modified organism. In another embodiment the absence in the sample of any of the plurality of target nucleic acid sequences is indicative of a bacterium, a virus, an insect, or a genetically-modified organism.

In yet another aspect of the invention devices are provided for detecting the presence, in a sample placed therein, of at least one target nucleic acid sequence, the devices comprise:

a reaction vessel into which the sample is introduced, the reaction vessel comprising a reaction mixture suitable for target sequence-dependent, primer-initiated, DNA polymerase-mediated nucleic acid amplification under conditions also permitting catalytic nucleic acid activity, the reaction mixture comprising the reactants for amplification of nucleic acids in the sample and a primer mixture comprising one or more primers sufficient for amplifying each of the at least one target nucleic acid sequences to be detected;

wherein for each of the at least one target nucleic acid sequences to be detected, there is at least one primer in said primer mixture comprising an antisense sequence of a catalytic nucleic acid such that a corresponding sense strand of said catalytic nucleic acid is incorporated into an amplicon produced when that target is present in the sample; the sense strand comprising an active catalytic nucleic acid that recognizes and modifies a corresponding substrate;

a support means for bearing the substrate for each catalytic nucleic acid activity corresponding to each of the at least one target nucleic acid sequences to be detected; wherein a detectable signal is produced upon modification of each such substrate by the catalytic nucleic acid.

The device in one embodiment is a simple dipstick, or a strip test, the basic form and structure of which are known in the art. FIG. 3 depicts a dipstick device adapted for use herein.

With reference to FIG. 3, an example of a “point of care” testing (e.g. a “dip stick”) is depicted. This shows a generalised format of an exemplary multiplex “dipstick” test, which can be provided as a kit, as a useful application of the methods provided herein. This reaction can be performed under isothermal conditions at any temperature suited to the particular methods.

Step (i) represents the amplification of each of several targets of interest in a sample. If the reaction tube remains translucent, for example, to the unaided eye, this indicates no amplification has occurred (negative amplification result for all targets). If visible turbidity develops, successful amplification has been achieved (e.g. positive amplification result for one or more targets).

Step ii depicts that the exact targets present in the positive samples can then be identified by exposure to the dipstick.

Step iii, in this example, shows five dual-labeled fluorescent substrates covalently attached to the dipstick, for example, at discrete locations. In this embodiment, the five substrates allow, for example, for the identification of one positive and one negative amplification control and three target sequences of interest. While the sequences of each substrate must be distinct from each other, the fluorophore/quencher dye pair or other detectable portion may be the same.

Step iv shows that active DNAzymes generated from the amplification of each target present in the sample will cleave the substrate corresponding to that target. Cleavage of each fluorescent substrate will result in removal of the quencher and concomitant fluorescence. For example, where the substrates are attached in discrete bands, the resulting banding pattern on the strip test could identify the target species in the test sample. Such a pattern could also be used, for example, as an indication for personalised therapy.

Other configurations for such dipstick devices are readily envisioned. For example, in one embodiment the dipstick has three spots—a positive control, a negative, and a spot wherein any of a plurality of target sequences can be detected. Thus in reading the device, the positive control must show a detectable signal, the negative control must not show a signal, and if the controls so read, then a detectable signal in the third spot would indicate the presence of at least one of the plurality (two or more here) of targets sequences are present in the sample.

FIG. 4 depicts a striptest adapted for use herein. The various embodiments of tests are not mutually exclusive and may be used together to provide, for example, an initial screening tool and a more detailed diagnostic tool capable of further distinguishing the target sequences present where needed.

With more particular reference to FIG. 4, additional exemplary applications of devices for use herein are depicted.

In Panel (a): It can be seen that the “dipstick test” concept can be extended to a variety of “Striptests”:

In step i, multiplexed amplification of one or more targets present in each of a panel of samples—each tube is used to amplify the one or more targets present in the sample,

In step ii, the amplicons, if any, produced in the presence of targets are transferred. The positive samples are transferred to the Striptest device, which contains covalently-attached, single-colour capture substrates, specific to each amplicon. The targets can be attached in arrays wherin for each sample the array is separated into separate channels, wells, areas, or other sample retention methods (not shown) to prevent intermixing of samples. The targets may be arrayed in discrete locations within each channel, well; or area, or the targets and controls may be arranged in a YES/NO or “stop light” fashion, as described above. In another embodiment the entire strip test is used to produce a more detailed array for a single sample with a large number of potential target sequences. The results after detection provide a unique pattern based on the targets present, such as can be seen with gene array, sequence chips, and the like.

In step iii, detection is performed. Target-specific amplification results in cleavage of the corresponding substrate with concomitant signal generation, preferably at a corresponding position. In the embodiment shown, each sample s contained within a channel, well or area. The results, preferably in a pattern of spots, lines, or bands, or other array for easy cognition, identify specific target sequences in a panel of patient samples, such as for viral screening of blood products from a variety of patients or sources.

With further reference to FIG. 4, Panel (b) exemplifies a multiplex amplification and detection or quantification that can be carried out in a homogeneous single vessel such as a microtube or microwell format. A single tube multiplexed reaction is conducted in step (i). Amplicons from each target amplified from a multiplex reaction harbour specific DNAzyme tag, which cleaves complementary substrate (Sub) labelled with distinct fluorophore (F). In step (ii), detection is performed. Successful target amplification from a multiplex reaction can be determined, for example, by the wavelength of the signal generated by specific substrate cleavage at the end of the reaction. The change in fluorescence from these multiplexed reactions can be monitored by end point or in real time.

The particular devices are unique in that they allow a target nucleic acid or a plurality thereof to be amplified and detected in a simple system that can be read visually, or with the aid of a device adapted therefor. Thus, in one embodiment each of a plurality of target nucleic acid sequences can be amplified and detected using the device.

In one embodiment each substrate is localized in a discrete location on the support means, and the detectable signal remains so localized during detection. In particular applications each substrate is covalently localized and the detectable signal remains covalently attached to the support means for detection after modification thereof. The localized substrate is cleaved by the catalytic nucleic acid. Preferably, the detectable signals are each distinct where it is desired to detect each of a plurality (2 or more) of target sequences. The devices are also adaptable for the detection of any of a plurality of target sequences as described herein—in which case there is no need for distinctly detectable substrate modification. Thus, in such embodiments the detectable signal produced from each substrate is not distinct, and the device detects the presence of any of a plurality of target nucleic acids.

In preferred embodiments the reaction vessel is a tube, a well, a chamber, or a fluidic channel. However, any vessel suitable for holding the reaction volume can be adapted for use herein.

In a preferred embodiment, the support means is a polymeric support, a membrane, a bead, a metallic surface, or a glass surface. In other examples, the support means is a surface that is chemically-modified to allow convenient attachment of a substrate thereto. Such support means are known in the art and are used, for example, for enzyme assays with immobilized substrates, in affinity chromatography and similar affinity purification techniques; nucleic acid transfer, membrane blotting techniques for proteins and nucleic acids, and the like. The skilled artisan can prepare such a support means without undue experimentation. In addition rapid diagnostic test kits are known in the art for a variety of purposes and the skilled artisan will appreciate that similar techniques can be used in preparing the support means for the substrate of the catalytic nucleic acid enzyme reaction.

In one embodiment the support means is in the form of a dipstick that can be at least partially inserted into the reaction vessel. In such a case, the amplification occurs in the reaction vessel and the dipstick is inserted into the reaction prior to, during or after the initiation of amplification (e.g. by the introduction of sample or reaction components which may be in separated compartments). As the amplification progresses, the active catalytic nucleic acid is produced on an amplicon and is available to cleave substrate on the dipstick. A distinctive pattern can be used to provide detailed results as to the presence or absence of specific target sequences, or a simple yes/no answer as to the presence or absence of the target can be accomplished via, for example a color change, or similar easy to detect signal. Preferably the devices comprise at least a negative control reaction (e.g. receiving no sample), and a positive control reaction (having e.g. a target nucleic acid present, or an active catalytic nucleic acid specific for a distinct substrate included in the device, for example as depicted in FIGS. 3 (for a dipstick) and 4 (for a strip-test). Thus, in one embodiment the negative and positive controls are also localized on the support means. The detectable signal can be any signal that can be detected by e.g. vision, or smell, but preferably comprises a colorometric signal, fluorescence, luminescence, turbidity, or radioactivity.

The devices can be established so as to allow nucleic acid amplification via PCR, SDA, RCA, LAMP, TMA, 3SR, or NASBA. In addition, the devices can be used either in a thermocycler where required, or more preferably can be incubated isothermally after the sample is added. The devices are also adapted for use in real-time monitoring applications and therefore preferably provide a change in a detectable signal that can be monitored in real-time.

The devices also preferably comprise a method that can be conducted under field conditions, in an office, or in a mobile laboratory. Accordingly, the devices while quite sensitive, are sufficiently robust in terms of reaction conditions and sample size that a proper or acceptable result can be obtained even where the conditions are not ideal or completely in agreement with a comparable assay in the laboratory. Preferably the devices are adapted to provide an acceptably low degree of false positives and an acceptably low degree of false negatives.

In another aspect of the present invention kits for practicing the methods are provided.

Thus, in one embodiment kits are provided for use in detecting the presence of a target nucleic acid sequence in a sample. The kits comprise:

(a) a primer mixture comprising:
(i) a pair of inner primers; or
(ii) a pair of inner primers and at least one outer primer; or
(iii) a pair of inner primers and at least one loop primer; or
(iv) a pair of inner primers, at least one outer primer, and at least one loop primer;

wherein the pair of inner primers comprises a forward inner primer and a backward inner primer, and each inner primer comprises a first portion that hybridizes to a sense sequence of a target nucleic acid sequence, and a second portion that hybridizes to an antisense sequence of the target nucleic acid sequence;

wherein each outer primer present hybridizes to a portion of the target nucleic acid sequence;

wherein each loop primer present comprises a portion complementary to a single stranded loop region on an amplicon produced from the extension of the forward inner primer or the backward inner primer;

wherein at least one primer in the primer mixture comprises an antisense sequence of a catalytic nucleic acid such that a corresponding sense strand of said catalytic nucleic acid is incorporated in an amplicon produced during amplification of that target nucleic acid;

wherein, when the primer mixture does not comprise any loop primers, the antisense sequence of a catalytic nucleic acid is positioned between the first and the second portion of one or both of the forward or backward inner primers; and

wherein, when the primer mixture comprises at least one loop primer the antisense sequence of a catalytic nucleic acid is positioned between the first and the second portion of one or both of the forward or backward inner primer, or at the 5′ end of one or more loop primers, or both positions; and

(b) a substrate modifiable by the catalytic nucleic acid and whose modification generates a detectable signal;
(c) a reaction mixture providing conditions permitting catalytic nucleic acid activity and target-dependent, primer-initiated, DNA polymerase-mediated nucleic acid amplification, and reactants required therefor; and

(d) a DNA polymerase having strand displacement activity.

In one embodiment the primer mixture comprises a pair of inner primers, but no outer primers or loop primers. In another, the primer mixture comprises a pair of inner primers and at least one outer primer, but no loop primers. In yet another the primer mixture comprises a pair of inner primers and at least one loop primer, but no outer primers. In another embodiment the primer mixture comprises a pair of inner primers, at least one outer primer, and at least one loop primer. In a preferred embodiment the primer mixture comprises at least a pair of inner, outer, and loop primers for each target to be detected in the method.

The kits provided herein may further comprise instructions for detecting the target nucleic acid, particularly in accordance with the methods provided herein.

In one embodiment, a kit comprises a reverse transcriptase or reagents for producing a DNA from an RNA target nucleic acid.

In another embodiment, kits are provided for use in detecting the presence of each of a plurality of target nucleic acid sequences in a sample. The kits comprise:

(a) a primer mixture comprising, for each of the plurality of target nucleic acid sequences to be detected at least:
(i) a pair of inner primers; or
(ii) a pair of inner primers and at least one outer primer; or
(iii) a pair of inner primers and at least one loop primer; or
(iv) a pair of inner primers, at least one outer primer, and at least one loop primer;

wherein each pair of inner primers comprises a forward inner primer and a backward inner primer, and each inner primer comprises a first portion that hybridizes to a sense sequence of at least one of the plurality of target nucleic acid sequences, and a second portion that hybridizes to an antisense sequence of that target nucleic acid sequence;

wherein each outer primer present hybridizes to a portion of at least one of the plurality of target nucleic acid sequences;

wherein each loop primer present comprises a portion complementary to a single stranded loop region on an amplicon produced from the extension of at least one forward inner primer or backward inner primer corresponding to at least one of the plurality of target nucleic acid sequences;

wherein for each of the plurality of target nucleic acid sequences, at least one primer in the primer mixture comprises an antisense sequence of a distinctly detectable catalytic nucleic acid such that a corresponding sense strand of said distinctly detectable catalytic nucleic acid is incorporated in an amplicon produced during amplification of that target nucleic acid sequence;

wherein for each of the plurality of target nucleic acid sequences, when the primer mixture does not comprise any loop primers for that target nucleic acid sequence, antisense sequence of a distinctly detectable catalytic nucleic acid is positioned between the first and the second portion of one or both of the forward and backward inner primers; and

wherein for each of the plurality of target nucleic acid sequences, when the primer mixture comprises at least one loop primer for that target nucleic acid sequence, the antisense sequence of a distinctly detectable catalytic nucleic acid is positioned between the first and the second portion of one or both of the forward and backward inner primers, or at the 5′ end of one or both of the loop primers, or both such locations;

(b) for each of the plurality of target nucleic acid sequences, a substrate modifiable by the catalytic nucleic acid corresponding to that target nucleic acid sequence, the modification of which substrate generates a distinctly detectable signal;

(c) a reaction mixture providing conditions permitting catalytic nucleic acid activity and target-dependent, primer-initiated, DNA polymerase-mediated nucleic acid amplification, and reactants required therefor; and

(d) a DNA polymerase having strand displacement activity.

In one embodiment the primer mixture comprises for each of the plurality of target sequences a pair of inner primers, but no outer primers or loop primers. In another, the primer mixture comprises, for each of the plurality of target sequences, a pair of inner primers and at least one outer primer, but no loop primers. In yet another the primer mixture comprises a pair of inner primers and at least one loop primer, but no outer primers for each of the plurality of target sequences. In another embodiment the primer mixture comprises a pair of inner primers, at least one outer primer, and at least one loop primer for each of the plurality of target sequences. In a preferred embodiment the primer mixture comprises at least a pair of inner, outer, and loop primers for each target to be detected in the method.

The kits provided herein may further comprise instructions for detecting the target nucleic acid, particularly in accordance with the methods provided herein.

In one embodiment, a kit comprises a reverse transcriptase and reagents as required for producing a DNA from an RNA target nucleic acid.

In another embodiment kits for use in detecting the presence of any of a plurality of target nucleic acid sequences in a sample are provided herein. The kits comprise:

(a) a primer mixture comprising one or more primers sufficient for amplifying each of the plurality of target nucleic acid sequences to be detected;

wherein for each of the plurality of target nucleic acid sequences, there is at least one primer in the primer mixture comprising an antisense sequence of a catalytic nucleic acid such that a corresponding sense strand of the catalytic nucleic acid is incorporated into an amplicon produced during amplification of that target nucleic acid sequence;

(b) for each of the plurality of target nucleic acid sequences, a substrate modifiable by the catalytic nucleic acid corresponding to that target nucleic acid sequence, the modification of which substrate generates a detectable signal;

(c) a reaction mixture providing conditions permitting catalytic nucleic acid activity and target-dependent, primer-initiated, DNA polymerase-mediated nucleic acid amplification, and reactants required therefor; and

(d) a DNA polymerase suitable for amplifying the target nucleic acid sequences.

In one embodiment, the kits comprise DNA polymerase which has strand displacement activity. The kits further comprise a primer mixture that comprises at least:
(i) a pair of inner primers; or
(ii) a pair of inner primers and at least one outer primer; or
(iii) a pair of inner primers and at least one loop primer; or
(iv) a pair of inner primers, at least one outer primer, and at least one loop primer;

wherein each pair of inner primers comprises a forward inner primer and a backward inner primer, and each inner primer comprises a first portion that hybridizes to a sense sequence of at least one of the plurality of target nucleic acid sequence, and a second portion that hybridizes to an antisense sequence of that target nucleic acid sequence;

wherein each outer primer present hybridizes to a portion of at least one of the plurality of target nucleic acid sequences;

wherein each loop primer present comprises a portion complementary to a single stranded loop region on an amplicon produced from the extension of at least one forward inner primer or backward inner primer corresponding to at least one of the plurality of target nucleic acid sequences;

wherein for each of the plurality of target nucleic acid sequences, when the primer mixture does not comprise any loop primers for that target nucleic acid sequence, the antisense sequence of a detectable catalytic nucleic acid is positioned between the first and the second portion of one or both of the forward and backward inner primers; and

wherein for each of the plurality of target nucleic acid sequences, when the primer mixture comprises at least one loop primer for that target nucleic acid sequence, the antisense sequence of a detectable catalytic nucleic acid is positioned between the first and the second portion of one or both of the forward and backward inner primers, or at the 5′ end of one or more of the loop primers, or both positions.

In one embodiment of the foregoing kit, the primer mixture comprises for each of the plurality of target sequences a pair of inner primers, but no outer primers or loop primers. In another, the primer mixture comprises for each of the plurality of target sequences, a pair of inner primers and at least one outer primer, but no loop primers. In yet another the primer mixture comprises a pair of inner primers and at least one loop primer, but no outer primers for each of the plurality of target sequences. In another embodiment the primer mixture comprises a pair of inner primers, at least one outer primer, and at least one loop primer for each of the plurality of target sequences. In one embodiment, there is a pair of inner, outer, and loop primers for each target sequence to be detected.

In any given kit herein disclosed the primer mixture may comprise a combination of different sets of primers (i), (ii), (iii), and/or (iv) above such that a different selection of primers is present for each of a plurality of primers. For example, some target sequences may be lacking corresponding loop or outer primer, some may be lacking both, and some may lack neither.

The kits described herein can also include reactants for nucleic acid amplification wherein the amplification method is PCR, SDA, RCA, LAMP, TMA, 3SR, or NASBA.

The kits provided herein may further comprise instructions for detecting the target nucleic acid, particularly in accordance with the methods provided herein.

In one embodiment, a kit comprises a reverse transcriptase and reagents, as required for producing a DNA from an RNA target nucleic acid.

In another of its several aspects, the invention provides novel DNA molecules: The molecules are particularly useful as primers for methods using the combined amplification and detection of nucleic acid sequences. The DNA molecules comprise:

at least a first portion complementary to at least a first portion of a target nucleic acid sequence,

a second portion complementary to an antisense sequence of a second portion of the target nucleic acid sequence, and

a third portion comprising an antisense sequence of a catalytic nucleic acid; said third portion positioned between the first and second portions of said DNA molecule.

In a preferred embodiment of the DNA molecules, the antisense sequence is the antisense sequence of a DNAzyme. In another embodiment, the antisense sequence is the antisense sequence of a ribozyme.

In yet another aspect of the present invention methods for the amplification and detection of at least one target nucleic acid sequence comprising using the DNA molecules are provided herein. The DNA molecules are used as primers during the amplification of the target nucleic acid sequence, wherein at least one amplicon produced during amplification comprises the sense strand of the catalytic nucleic acid, and wherein the detection comprises the modification of at least one detectable substrate by the catalytic nucleic acid in the at least one amplicon.

Preferably the methods involve amplification that is isothermal. More preferably the isothermal methods are conducted at a temperature less than about 62° C., but above ambient temperature. More preferably temperatures of about 37° C. to about 56° C. are used for isothermal amplification.

In one embodiment the methods encompass amplification methods which comprise the use of a DNA polymerase with strand displacement activity. In certain preferred methods, the target nucleic acid is RNA and the method comprises the additional step of reverse transcribing the RNA into DNA prior to amplification.

In one embodiment the modification of the detectable substrate is cleavage, and the method further comprises the step of using a plurality of cleavable substrates, the cleavage of each of which is distinctly detectable, wherein there is one such substrate for each of the plurality of target nucleic acid sequences.

It is to be understood that the figures and examples provided herein are to exemplify various aspects of the invention, and not to limit the invention in any of its various embodiments. The skilled artisan will appreciate that the examples and the related description can not encompass the entirety of the invention, and that aspects of the invention are capable of variation and alteration, keeping in mind the language, intent, and spirit of the claims that are appended hereto, which also can provide an understanding of various aspects of the invention.

### EXAMPLES

### Example 1

### Detection of Lambda DNA

A. DNA Oligonucleotides

Six LAMP primers (L-FIP, L-BIP, L-Outer F3, L-Outer B3, L-Loop B, and L-Loop F) with homology to lambda DNA, and suitable for amplification of lambda DNA were synthesized by Trilink BioTechnologies. The sequences of these primers were previously published (Nagamine et al., 2002).

The inner L-FIP primer consisted of anti-sense F1 sequence (20 nt) and sense F2 sequence (26 nt). The inner L-BIP primer consisted of the anti-sense B1 (26 nt) and sense B2 sequence (25 nt). The outer primers were sense F3 sequence (22 nt), and anti-sense B3 (22 nt) respectively. The L-Loop F (17 nt) and L-Loop B (20 nt) sequences were anti-sense, and sense, respectively, as shown below.

A primer was designed based on the L-Loop B primer by incorporating an antisense sequence of a DNAzyme at the 5′ end. The antisense sequence is an inactive sequence complementary to the catalytically-active DNAzyme. This primer, designated L-cDzX/Loop B, was also synthesized.

Sequences are shown below in the 5′-3′ orientation.

Inner L-FIP primer:

(SEQ ID NO:1)

5′-CAGCCAGCCGCAGCACGTTCGCTCATAGGAGATATGGTAGAGCCGC-
3′
Inner L-BIP primer:

(SEQ ID NO:2)

5′-GAGAGAATTTGTACCACCTCCCACCGGGCACATAGCAGTCCTAGGGA
CAGT-3′
Outer L-F3 Primer:

(SEQ ID NO:3)

5′-GGCTTGGCTCTGCTAACACGTT-3′
Outer L-B3 Primer:

(SEQ ID NO:4)

5′-GGACGTTTGTAATGTCCGCTCC-3′
L-Loop F Primer:

(SEQ ID NO:5)

5′-CTGCATACGACGTGTCT-3′
L-Loop B Primer:

(SEQ ID NO:6)

5′-ACCATCTATGACTGTACGCC-3′
L-cDzX/Loop B Primer:

(SEQ ID NO:7)

5′ AAGGTTTCCTCTCGTTGTAGCTAGCCTCCCTGGGCACAGCGACTCAC
CATCTATGACTGTACGCC-3′

B. Reporter Substrate

The reporter substrate (Sub X) was synthesized by Trilink BioTechnologies. Sub X, shown below, is a chimeric molecule containing both RNA (shown in lower case) and DNA bases. The 3′ terminus cannot be extended by polymerase during amplification.

Substrate Sub X:

(SEQ ID NO:8)

5′-AAGGTTTCCTCguCCCTGGGCA-3′

Sub X for these experiments was synthesized with a detectable substituent (6-carboxyfluorescin (“6-FAM”)) attached to the 5′ terminus, and a quenching substituent (Black Hole Quencher (“BHQ1”)) attached to the 3′ terminus. The cleavage of the reporter substrate was monitored at 530 nm (emission wavelength) with excitation at 485 nm (excitation wavelength).

C. Template DNA

Lambda DNA was purchased from New England Biolabs for use as target template.

D. Amplification and Detection of Lambda DNA

Lambda DNA was amplified as follows: Lambda DNA at different copy numbers was provided in the reactions (1 to 10⁷ copies per reaction). The reaction mixtures also contained the following primers: 0.8 μM of inner L-FIP primer, 0.8 μM of inner L-BIP primer, 0.2 μM of outer L-F3 primer, 0.2 μM of outer L-B3 primer, 0.4 μM L-Loop F primer, 0.2 μM L-Loop B primer, and 0.2 μM L-cDzX/Loop B primer. Also included were 0.4 μM of the substrate Sub X, 400 μM dNTPs (each of dATP, dCTP, dTTP, and dGTP), 0.8× thermopol buffer (16 mM Tris at pH 8.8 at 25° C., 8 mM KCl, 1.6 mM MgSO₄, 0.08% Triton, and 8 mM NH₄SO₂), 20 mM NaCl, 2 mM MgCl₂, 1 mM MgSO₄, and 8 units of Bst DNA polymerase (New England Biolabs). The total reaction volume was 25 μl. During amplification, amplicons containing the target lambda sequences as well as catalytically active (sense) copies of the DNAzyme were produced. The active DNAzyme is designed to cleave the RNA/DNA reporter substrate included in the reactions.

The negative control reactions contained all reaction components with the exception of lambda DNA. The reactions were incubated at 56° C. for 70 minutes in an ABI 7700 Sequence Detection System (Applied Biosystems).

Fluorescence was measured throughout the amplification reaction to monitor the accumulation of lambda-containing amplicons. An increase in fluorescence at 530 nm over that in the negative controls was observed. The increase over time was dependent on the initial copy number of the lambda DNA in the reaction. The results are shown in Table 1-1 and in FIG. 5.

TABLE 1-1
Time to detection of lambda DNA as a function of copy number.

| Copy Number | Time (min)* | Results |
|---|---|---|
| 10⁷ | 17.1 | Calibration curve (average of |
| 10⁶ | 19.8 | triplicate reactions) |
| 10⁵ | 22.6 | R² = 0.998; |
| 10⁴ | 25.4 | Slope = −2.8 |
| 10³ | 28.3 | |
| 10² | 31.8 | Detectable |
| 10 | 39.6 | Limit of detection |
| 1 | No signal | Not detected |
| 0 (no template | No signal | 3 negative controls - no signal at |
| control) | | 70 min |

*Baseline was monitored from time 1-10 min; the Threshold was set at 0.4.

These results established that homogeneous amplification in a simple format that allows real-time detection via fluorescence can be accomplished. As exemplified, the method allows detection of as few as about 10 copies of a target nucleic acid in a sample.

### Example 2

### Specific Detection of Lambda DNA Against a Background of Unrelated DNA

A. Primers and Substrate:

The DNA oligonucleotides, and the reporter substrate were as in Example 1.

B. Template DNA

Lambda DNA was purchased from New England Biolabs for use as target template. The lambda DNA was diluted in a background of genomic DNA from the human cell line CEM-T4 (cells obtained from the “NIH AIDS Research & Reference Reagent Program”). Genomic DNA from the CEM-T4 cells was prepared using the DNeasy Tissue extraction kit (Qiagen).

C. Amplification and Detection of Lambda DNA in a Background of Unrelated DNA

Lambda DNA was amplified as follows: Lambda DNA at different copy numbers was provided in the reactions (10¹ to 10⁸ copies per reaction). A background of 100 ng of CEM-T4 genomic DNA was included. The reaction mixtures also contained the following primers: 0.8 μM of inner L-FIP primer, 0.8 μM of inner L-BIP primer, 0.2 μM of outer L-F3 primer, 0.2 μM of outer L-B3 primer, 0.4 μL-Loop F primer, 0.2 μM L-Loop B primer, and 0.2 μM L-cDzX/Loop B primer. Also included were 0.4 μM of the substrate Sub X, 400 μM dNTPs (each of dATP, dCTP, dTTP, and dGTP), 0.8× thermopol buffer (16 mM Tris at pH 8.8 at 25° C., 8 mM KCl, 1.6 mM MgSO₄, 0.08% Triton, and 8 mM NH₄SO₂), 20 mM NaCl, 2 mM MgCl₂, 1 mM MgSO₄, and 8 units of Bst DNA polymerase (New England Biolabs). The total reaction volume was 25 μl. During amplification, amplicons containing the target lambda sequences as well as catalytically active (sense) copies of the DNAzyme were produced. The active DNAzyme is designed to cleave the RNA/DNA reporter substrate included in the reactions.

Negative control reactions lacking only the lambda DNA or lacking any added DNA (6 CEM-T4 controls and 6 no DNA (H₂O) controls) were performed in parallel. All reactions were incubated at 56° C. for 50 minutes in an ABI 7700 Sequence Detection System (Applied Biosystems).

Fluorescence was measured throughout the amplification reaction to monitor the accumulation of lambda-containing amplicons. An increase in fluorescence at 530 nm over that in the negative controls was observed. The increase was dependent on the initial copy number of the lambda DNA in the reaction. The results are shown in Table 2-1 and in FIG. 6.

These results confirm that the methods provided herein are adaptable for amplification and detection of a specific target DNA in a background of unrelated DNA in a simple format that allows real time detection. As few as about 1000 copies were detected against a vast background of unrelated sequences.

TABLE 2-1
Time to detection of lambda DNA in a background of excess
unrelated human DNA, as a function of copy number.

| Copy Number | Time (min)* | Results |
|---|---|---|
| 10⁸ | 21.2 | Calibration curve (average of |
| 10⁷ | 24.1 | triplicate reactions) |
| 10⁶ | 27.0 | R² = 0.990; |
| 10⁵ | 30.0 | Slope = −3.142 |
| 10⁴ | 34.0 | |
| 10³ | 36.6 | |
| 10² | No signal | Not Detected |
| 10 | No signal | Not Detected |
| 0 (no template | No signal | 12 negative controls (6 H₂0 |
| control) | | and 6 CEMT (100 ng)) |
| | | No signal at 50 mins. |

*The Baseline was monitored from time 1-10 mins; the Threshold was set at 0.5.

### Example 3

### Comparison of Efficiencies of Different Ratios of cDzX/Loop B Primer to Loop B Primer

A. Primers and Substrate:

The DNA primers (L-FIP, L-BIP, Outer L-F3, Outer L-B3, L-Loop B, L-cDzX/LoopB and L-Loop F) and the reporter substrate (Sub X) were as in Example 1.

Lambda DNA was amplified as follows: Each reaction received 5 pg lambda DNA template. The reaction mixtures each contained the following primers: 0.8 μM of inner L-FIP primer, 0.8 μM of inner L-BIP primer, 0.2 μM of outer L-F3 primer, 0.2 μM of outer L-B3 primer, 0.4 μM L-Loop F primer. Each reaction also included 0.4 μM Sub X, 400 μM dNTPs (each of dATP, dCTP, dTTP, and dGTP), 0.8×thermopol buffer (16 mM Tris at pH 8.8 at 25° C., 8 mM KCl, 1.6 mM MgSO₄, 0.08% Triton, and 8 mM NH₄SO₂), 20 mM NaCl, 2 mM MgCl₂, 1 mM MgSO₄, 0.2×NEB 3 (10 mM Tris-HCl pH 7.9 at 25° C., 2 mM MgCl₂, 20 mM NaCl, 0.2 mM dithiolthreitol), and 8 units of Bst DNA polymerase (New England Biolabs).

Different amounts of L-Loop B and L-cDzX/Loop B primers were provided in the reactions as follows: (1) 0.4 μM L-cDzX/Loop B primer, no L-Loop B primer; (2) 0.2 μM L-cDzX/Loop B primer, no L-Loop B primer; and (3) 0.2 μM L-cDzX/Loop B primer plus 0.2 μM L-Loop B primer. The total reaction volume was 25 μl. Each reaction was conducted in triplicate.

During amplification, amplicons containing the target lambda sequences as well as catalytically active (sense) copies of the DNAzyme were produced. The active DNAzyme is designed to cleave the SubX reporter substrate included in the reactions. Signal generated as a result of such DzX-mediated cleavage of SubX was detected using an ABI Prism 7700. Data were collected at 1 minute intervals over a time period of 1.5 hours at 56° C.

Data analysis using a threshold of 0.5 showed the average time to reach threshold fluorescence for each of reactions conditions as follows:

| | |
|---|---|
| (1) 0.4 μM L-cDzX/Loop B: | 49.2 minutes; |
| (2) 0.2 μM L-cDzX/Loop B: | 44.6 minutes; and |
| (3) 0.2 μM L-cDzX/Loop B plus 0.2 μM L-Loop B | 36.8 minutes. |

The results are shown in graphical form in FIG. 7. These data provide evidence that maintaining both L-cDzX/Loop B and L-Loop B primers in the reaction, for example with total L-Loop B at 0.4 μM, provides improved signal generation compared to amplification where the L-Loop B primer is comprised entirely of the tagged primer at either 0.4 μM or 0.2 μM. In this example, where analysis was carried out using a threshold of 0.5, the 0.2 μM L-Loop B plus 0.2 μM L-cDzX/Loop B provided detection at least 7 minutes earlier than either 0.2 μM or 0.4 μM of L-cDzX/Loop B primer in the absence of L-Loop B primer.

### Example 4

### Comparison of Methods Using L-cDzX/Loop B Primer with L-cDzX/BIP Primer

A. Primers and Substrate:

The DNA primers (L-FIP, L-BIP, Outer L-F3, Outer L-B3, L-Loop B, L-cDzX/LoopB and L-Loop F) and the reporter substrate (Sub X) were as in Example 1.

It was determined that the cDz tag (the antisense sequence of the catalytic nucleic acid) could be incorporated into (1) the 5′ end of either the Loop B or the Loop F primers, or (2) between the F1c and F2 domains of the FIP primer or the B1c and B2 domains of BIP primer. Primers were therefore designed and tested to generally compare the primers incorporated at the 5′ end of either of the loop primers (“Model A”) to those incorporated between the domains or portions of either of the pair of inner primers (“Model B”).

The L-cDzX/BIP primer was designed based on the L-BIP primer by incorporating an antisense sequence between the B1c and B2 domains or portions of the L-BIP primer. The antisense sequence is an inactive sequence complementary to a catalytically-active DNAzyme. This primer was also synthesized. The sequence is shown below in the 5′-3′ orientation.

L-cDzX/BIP primer:

(SEQ ID NO:9)

5′ GAGAGAATTTGTACCACCTCCCACCGAAGGTTTCCTCTCGTTGTAGC
TAGCCTCCCTGGGCAGGCACATAGCAGTCCTAGGGACAGT-3′

Reactions

Reactions contained 5 pg of lambda DNA (10⁴ copies). All reactions were incubated in a total reaction volume of 25 μl in common reaction mixture as follows: 0.4 μM of Sub X, 400 μM dNTPs (each of dATP, dCTP, dTTP, dGTP), 0.8×thermopol buffer (16 mM Tris pH 8.8 at 25° C., 8 mM KCl, 1.6 mM MgSO₄, 0.08% Triton, and 8 mM NH₄SO₂), plus 20 mM NaCl, 2 mM MgCl₂, 1 mM MgSO₄, with 8 units of Bst DNA polymerase (New England Biolabs). The common primers in each reaction were 0.8 μM of L-FIP, 0.2 μM of L-F3, 0.2 μM of L-B3, and 0.4 μM L-Loop F. Additional primers were added to specific reactions as follows:

Reactions Using L-cDzX/LoopB Primer (Model A):

0.8 μM of L-BIP, 0.2 μM L-Loop B, 0.2 μM L-cDzX/LoopB.

Reactions Using L-cDzX/BIP Primer (Model B):

0.4 μM of L-BIP, 0.4 μM of L-cDzX/BIP, 0.4 μM L-Loop B.

Each reaction was incubated at 56° C. for a period of 1.5 hours. Data from the emission spectra at 530 nm was collected at 1 minute intervals in an ABI Prism 7700 Detection System.

The fluorescence intensity for reactions using the L-cDzX/Loop B primer (Model A) peaked at 22300 fluorescence units after 45 minutes. The peak fluorescence intensity for the reactions using the L-cDzX/BIP primer (Model B), was 6400 fluorescence units at 1.5 hours. Thus, using the L-cDzX/Loop B primer gave a 3.5 fold greater signal than the L-cDzX/BIP primer, and produced signal 45 minutes earlier (about half the time). The results showing the time to reach the threshold level of fluorescence are provided in Table 4-1 and in FIG. 8.

TABLE 4-1
Time to detection using primers with the antisense DNAzyme
in either the Loop primers (cDzX/Loop B - Model A)
or the Inner primers (cDzX/BIP - Model B).

| Model | *Time (min). |
|---|---|
| Model A replicate 1 | 25.02 |
| Model A replicate 2 | 25.06 |
| Model B replicate 1 | 54.35 |
| Model B replicate 2 | 52.31 |

The Baseline was monitored from time 1-15 min; the Threshold was set at 0.85.

These results demonstrate that it is possible to generate a fluorescent signal following LAMP amplification of specific nucleic acid target using primers which contain the anti-sense DNAzyme placed either (i) within an inner primer (such as the L-cDzX/BIP primer) or (ii) at the 5′ end of a loop primer (such as the L-cDzX/Loop B). In this example, a higher level of fluorescence was generated when the antisense DNAzyme was placed on the Loop primer as compared to the inner BIP primer. Further, the signal was generated more rapidly when the antisense DNAzyme was placed on the Loop primer as compared to the inner BIP primer.

### Example 5

### Detection and Quantitation of PSA RNA in Total RNA Extracted from LnCap Cells in a Single Tube

Primers were designed for human prostate specific antigen (PSA). The primers for amplifying the PSA target were synthesized by TriLink Biotechnologies. Primer sequences are shown below in the 5′-3′ orientation.

PSA F3:

(SEQ ID NO:10)

5′-TGCTTGTGGCCTCTCGTG-3′
PSA B3:

(SEQ ID NO:11)

5′-GGGTGTGTGAAGCTGTG-3′
PSA FIP:

(SEQ ID NO:12)

5′-TGTTCCTGATGCAGTGGGCAGCTTTAGTCTGCGGCGGTGTTCTG-3′
PSA BIP:

(SEQ ID NO:13)

5′-TGCTGGGTCGGCACAGCCTGAAGCTGACCTGAAATACCTGGCCTG-
3′
PSA LoopF:

(SEQ ID NO:14)

5′-GACCCACTGAGGAGGCAC-3′
PSA LoopB:

(SEQ ID NO:15)

5′-TTTCATCCTGAAGAC-3′
PSA cDzY/LoopF:

(SEQ ID NO:16)

CAGCACAACCTCGTTGTAGCTAGCCTCACCAACCGGACCCACTGAGGAGG
CAC

Substrate SubY

A reporter substrate, SubY was used to detect PSA in this example. SubY was synthesized by Trilink BioTechnologies. Like SubX, SubY is a chimeric molecule containing both RNA (shown below in lower case) and DNA nucleotides (shown in upper case). As with SubX, the 3′ terminus of SubY as used herein cannot be extended by the polymerase during amplification. SubY was synthesized with a fluorescent substituent (6-carboxyfluorescin (“6-FAM”)) attached to the 5′-terminal nucleotide, and a quencher substituent (Black Hole Quencher (“BHQ1”)) attached to the 3′-terminal nucleotide. The cleavage of the Sub Y was monitored at 530 nm (FAM emission wavelength) with excitation at 485 nm.

The sequence of Sub Y was as follows:

SubY substrate:

(SEQ ID NO:17)

5′-CAGCACAACCguCACCAACCG-3′

Reactions

Reactions were conducted with different amounts of total LnCap RNA (800 ng, 160 ng, 32 ng, 6 ng, 1.28 ng, and 0 ng, respectively)

Each reaction was incubated with the following reagents: Primers (as shown above): 0.8 μM of PSA FIP, 0.8 μM of PSA BIP, 0.2 μM of PSA F3, 0.2 μM of PSA B3, 0.2 μM PSA loop F, 0.2 μM PSA cDzY/Loop F, and 0.4 μM PSA loop B. Also provided were 0.4 μM of Sub Y, 400 μM dNTPs (each of dATP, dCTP, dTTP, and dGTP), 0.8× Thermopol buffer (16 mM Tris pH 8.8 at 25° C., 8 mM KCl, 1.6 mM MgSO₄, 0.08% Triton and 8 mM NH₄SO₂) plus 0.2×NEB 3 buffer (10 mM Tris-HCl, 2 mM MgCl₂, 20 mM NaCl, 0.2 mM dithiolthreitol), 1×Rox reference dye, 8 units of Bst DNA polymerase (New England Biolabs) and 20 Units of MMLV-RT. The total reaction volume was 25 μl.

The mix was incubated at 50° C. for 30 minutes, followed by 90 minutes at 54° C. During the latter incubation only, data were collected at one minute intervals using an ABI Prism 7700.

The results, in Table 5-1, show the time to reach the threshold level of fluorescence.

TABLE 5-1
Time to detection for various amounts of RNA target.

| LNCap total | | | |
|---|---|---|---|
| RNA (ng) | | Time (min)* | Results |
| 800 | ng | 25.03 | Calibration curve |
| 160 | ng | 30.25 | (triplicate reactions) |
| 32 | ng | 34.24 | R² = 0.936; Slope = −8.513 |
| 6 | ng | 43.73 | |
| 1.28 | ng | >45 | Detectable |

| | | |
|---|---|---|
| No RNA | No signal | No signal in absence of |
| | | template RNA |

*Baseline was monitored from 1-15 min; the Threshold was set at 0.2.

As can be seen from the results, detection of PSA mRNA in as little as about a nanogram of total RNA from LnCAP cells was possible using the method, which combined reverse transcription, amplification, and detection in a single tube assay. The method was also amenable to real-time detection—in this case of human transcripts

### Example 6

### HEG Spacers and 5′ Self-Complementary Domains on cDz/Loop Primers

Primers: The primers used were as in Example 5.

The PSA cDzY/LoopF primer was also synthesised with an additional domain at the 5′ end. This additional domain consisted of a replication blocking hexaethylene glycol (HEG) spacer separating a 5′ sequence of bases designed to hybridise to an internal region on the primer consisting of part of the active core of the antisense DNAzyme along with some of the flanking sequence.

The HEG residue was predicted to block polymerase activity, resulting in the production of amplicons that have a 5′ overhang consisting of the base sequence 5′ of the HEG residue. The 5′ sequence was designed to hybridise to part of the antisense DNAzyme, forming an intramolecular hairpin. This was designed to increase the activity of the DNAzyme in the amplicon by making it more accessible for substrate binding and cleavage.

Primer PSA cDzY/LoopF was as above. PSA cDzY/LoopF 23/29, and PSA cDzY/LoopF 23/29 HEG are shown below. Primers PSA cDzY/LoopF and PSA cDzY/LoopF 23/29 differ only in the addition of a 5′ sequence of 7 bases complementary to part of the antisense DNAzyme. Primer PSA cDzY/LoopF HEG 23/29 is identical in sequence to PSA cDzY/LoopF 23/29 except that a HEG monomer is incorporated between the antisense DNAzyme and the additional 5′ sequence of 7 bases.

Primer PSA cDzY/LoopF/23/29:

(SEQ ID NO: 18)

GTGAGGCCAGCACAACCTCGTTGTAGCTAGCCTCACCAACCGGACCCACT
GAGGAGGCAC
Primer PSA cDzY/LoopF HEG 23/29:

(SEQ ID NO: 19)

GTGAGGC(HEG)CAGCACAACCTCGTTGTAGCTAGCCTCACCAACCGGAC
CCACTGAGGAGGCAC

Substrate SubY

The reporter substrate, SubY, as described in Example 5 was used here for detection of PSA amplification.

Reactions

The three primers, PSA cDzY/LoopF, PSA cDzY/LoopF 23/29, and PSA cDzY/LoopF HEG 23/29 were compared for performance in a reaction designed to provide a combination of amplification and detection. Reactions contained PSA cDNA (80 ng RNA equivalents) generated from total RNA extracted from the cancer cell line LnCap. Each reaction contained either PSA cDzY/LoopF, PSA cDzY/LoopF 23/29, or PSA cDzY/LoopF HEG 23/29 at 0.2 μM. The additional primers in each reaction were 0.4 μM of PSA FIP, 0.4 μM of PSA BIP, 0.2 μM of PSA F3, 0.2 μM of PSA B3, 0.2 μM PSA loop F, and 0.4 μM PSA loop B. The reaction mixture also included 0.4 μM of SubY, 400 μM dNTPs (each of dATP, dCTP, dTTP, and dGTP), 0.8× thermopol buffer (16 mM Tris pH 8.8 at 25° C., 8 mM KCl, 1.6 mM MgSO₄, 0.08% Triton, and 8 mM NH₄SO₂) plus 0.2×NEB 3 buffer (10 mM Tris-HCl, 2 mM MgCl₂, 20 mM NaCl, 0.2 mM dithiolthreitol), 1×Rox reference dye, and 8 units of Bst DNA polymerase (New England Biolabs). The total reaction volume was 25 μl. The reactions were incubated isothermally at 56° C. for 90 minutes in an ABI Prism 7700 system. Data points were collected at 1-minute intervals throughout the entire incubation period.

No signal was evident following an 80 minute incubation in the absence of template cDNA. The reactions using the primer PSA cDzY/LoopF HEG 23/29 showed a steeper amplification plot compared to those obtained with either the PSA cDzY/LoopF or PSA cDzY/LoopF 23/29 primers. The fluorescence plateau of the reactions containing PSA cDzY/LoopF HEG 23/29 primer was substantially higher than that of the PSA cDzY/LoopF or PSA cDzY/LoopF 23/29 primers.

The results are shown in Table 6-1.

TABLE 6-1
Performance of various primer designs

| | | Plateau level of fluorescence |
|---|---|---|
| Primer Name | Ct (mins) | (raw fluorescence units) |
| PSA cDzY/LoopF | 35.7 | 5000 |
| PSA cDzY/LoopF/23/29 | 42.5 | 3500 |
| PSA cDzY/LoopF HEG 23/29 | 34.5 | 9000 |

These results suggest that it is possible to improve the amplitude and/or the speed of fluorescent signal generation during amplification by using modified primers which incorporate HEG monomers and additional sequences capable of forming intra-molecular bonds within the primer. In this example, the introduction of bases capable of forming intra-molecular bonds within the primer alone delayed the generation of fluorescence and reduced the final fluorescence. However, when the bases capable of forming intra-molecular bonds were combined with a HEG monomer the fluorescent signal was generated more rapidly than with primers lacking these inclusions, and further, the final fluorescence was substantially higher.

### Example 7

### Detection and Quantification of Human Mammaglobin (MGB)

Primers with homology to human mammaglobin were synthesised by Trilink Biotechnologies. Primer sequences were as follows. There are 10 additional bases complementary to part of the antisense DNAzyme at the 5′ end of the primer. HEG=hexaethylene glycol monomer.

MGB F3:

(SEQ ID NO:20)

TCCTTGATCCTTGCCACC
MGB B3:

(SEQ ID NO:21)

AACATTCCTTCAATTCATCTA
MGB FIP:

(SEQ ID NO:22)

CCAGAGCCTGCGTAGCAGTGCTAGCAGCAGCCTCACCATGAA
MGB BIP:

(SEQ ID NO:23)

AATGTGATTTCCAAGACAATCAATCCGGCATTTGTAGTGGCATTCTC
MGB LoopF:

(SEQ ID NO:24)

AGCATCAGGACCATCAGCAA
MGB LoopB:

(SEQ ID NO:25)

AAGTGTCTAAGACTGAATACAAA
MGB cDzX/LoopF HEG 20/29:

(SEQ ID NO:26)

GTGAGGCTAG(HEG)CAGCACAACCTCGTTGTAGCTAGCCTCACCAACCG
AGCATCAGGACCATCAGCAA

Human Adult Normal Breast Total RNA was purchased from Biochain Institute, Inc. The total RNA was used in the synthesis of cDNA as follows: 2 μg of Human Adult Normal Breast Total RNA were incubated at 42° C. for 1 hour in a 20 μl reaction containing 1×PCR Buffer II (ABI), 5 mM MgCl₂, 1 mM dNTPs (each of dATP, dTTP, dGTP, and dCTP), 2.5 μM random hexamers (Roche), 40 U rRNAsin (Promega), and 100 U M-MLV RT (Promega).

Human Adult Normal Breast cDNA was then amplified in reactions containing either 500 ng, 100 ng, 20 ng, 4 ng, 800 pg, or 160 pg Breast cDNA. Control reactions with no cDNA were also included. Each reaction had the following primers: 0.8 μM of MGB FIP, 0.8 μM of MGB BIP, 0.2 μM of MGB F3, 0.2 μM of MGB B3, 0.4 μM MGB LoopB, 0.2 μM MGB LoopF, and 0.2 μM MGB cDzX/LoopF HEG 20/29. Reactions also contained 0.4 μM of Sub X, 500 μM dNTPs (each of dATP, dCTP, dTTP, and dGTP), 0.8× thermopol buffer (16 mM Tris pH 8.8 at 25° C., 8 mM KCl, 1.6 mM MgSO₄, 0.08% Triton, and 8 mM NH₄SO₂), 0.2×NEB3 Buffer (10 mM Tris-HCl, 2 mM MgCl₂, 20 mM NaCl, 0.2 mM dithiolthreitol) (New England Biolabs), and 8 units of Bst DNA polymerase (New England Biolabs) in a total reaction volume of 25 μl.

Reactions were incubated isothermally at 55° C. in an ABI 7700 for 90 minutes. Data were collected at one minute intervals. The results are shown in Table 7-1. It can be seen that the human mammaglobulin could be specifically detected within less than 1 hour, in all amounts of cDNA tested, even down to about 160 pg of total RNA equivalent.

TABLE 7-1
Time to detection threshold for human mammaglobulin in a
population of cDNA from total cellular RNA.

| Breast cDNA | | | |
|---|---|---|---|
| (Total RNA | | | |
| equivalent) | | Time (min)* | Results |
| 500 | ng | 31.13 | Calibration curve (average of |
| 100 | ng | 34.66 | duplicate reactions) |
| 20 | ng | 38.01 | R² = 0.87; |
| 4 | ng | 39.64 | Slope = −5.99 |
| 800 | pg | 49.6 | |
| 160 | pg | >52 | Detectable |

| | | |
|---|---|---|
| No cDNA | No signal | Six negative controls produced |
| | | no signal following 90 mins |
| | | incubation. |

*The Baseline was monitored from 1-15 mins; the Threshold was set at 0.5.

### Example 8

### Detection of Two Targets in a Duplex Amplification Reaction

Duplex amplification reactions were performed for the simultaneous detection of prostate specific antigen cDNA (PSA cDNA) and lambda genomic DNA.

Lambda genomic DNA was purchased from New England Biolabs.

PSA cDNA were synthesised from total RNA of LnCap cells. The total RNA was used in the synthesis of cDNA as follows: 2 μg of Human Total RNA from LnCap cells were incubated at 42 C for 1 hour in a 20 μl reaction containing 1×PCR Buffer II (ABI), 5 mM MgCl₂, 1 mM dNTPs (each of dATP, dTTP, dGTP, and dCTP), 2.5 μM random hexamers (Roche), 40 U rRNAsin (Promega), and 100 U M-MLV RT (Promega).

The duplexed reaction exemplified herein used two sets of oligonucleotide primers, each set specific for amplifying one of the target sequences, and each set comprising an antisense sequence for a different catalytic nucleic acid. Two substrates were used—each labelled with a different fluorophore and cleavable by only one of the catalytic nucleic acids.

Primer sequences for lambda DNA were the same as those listed in Example 1, while those for PSA DNA are described in Examples 5 and 6.

The reporter substrate for lambda (SubX, SEQ ID NO:8) was synthesized by Trilink BioTechnologies. As described in Example 1, SubX is a chimeric molecule containing both RNA and DNA nucleotides. The 3′ terminus of SubX as used herein, cannot be extended by the polymerase during amplification. For these experiments, SubX was synthesized with a fluorescent substituent (here, 6-carboxyfluorescein (“6-JOE”) was used) on the 5′ terminus, and a quencher substituent (here, Black Hole Quencher (“BHQ1”) was used) on the 3′ terminus. The cleavage of the reporter substrate for these experiments was monitored at 556 nm (JOE emission wavelength).

The reporter substrate for PSA (SubY) was synthesized by Trilink BioTechnologies. As described in Example 5, SubY is a chimeric molecule containing both RNA and DNA nucleotides. As with SubX, the 3′ terminus of SubY as used herein can not be extended by the polymerase during amplification. SubY was synthesized with a fluorescent substituent (here, 6-carboxyfluorescin (“6-FAM”) was used as it is distinguishable from JOE) attached to the 5′-terminal nucleotide, and a quencher substituent (here, Black Hole Quencher (“BHQ1”) was used) attached to the 3′-terminal nucleotide. The cleavage of Sub Y was monitored at 530 nm (FAM emission wavelength) with excitation at 485 nm (FAM excitation wavelength). The sequence of SubY is SEQ ID NO:17.

The reactions contained 5 pg of lambda DNA and 80 ng of PSA cDNA (total RNA equivalent). The primers included in the reaction were 0.4 μM lambda FIP, 0.4 μM lambda BIP, 0.2 μM L-F3, 0.2 μM L-B3, 0.4 μM L-LoopF, 0.2 μM L-LoopB, 0.2 μM L-cDzX/LoopB, 0.8 μM PSA FIP, 0.8 μM PSA BIP, 0.2 μM PSA F3, 0.2 μM PSA B3, 0.4 μM PSA LoopB, 0.3 μM PSA LoopF, and 0.1 μM PSA cDzY/LoopF HEG 23/29. The respective substrates were included for both lambda and PSA, i.e., 0.4 μM SubX, and 0.4 μM SubY. The reaction mixture also included, 500 μM dNTPs (each of dATP, dCTP, dTTP, and dGTP), 0.8× thermopol buffer (16 mM Tris pH 8.8 at 25° C., 8 mM KCl, 1.6 mM MgSO₄, 0.08% Triton, and 8 mM NH₄SO₂), 0.2×NEB3 Buffer (10 mM Tris-HCl, 2 mM MgCl₂, 20 mM NaCl, and 0.2 mM dithiolthreitol) (New England Biolabs), and 8 units of Bst DNA polymerase (New England Biolabs). The total reaction volume was 25 μl. Reactions were incubated isothermally at 56° C. for 3 hours on an ABI 7700 system. Data points were collected every minute. The results are shown in Table 8-1.

TABLE 8-1

| Target | Results |
|---|---|
| PSA cDNA | Increase in fluorescence at 530 nm following 1 hour |
| | incubation |
| Lambda DNA | Increase in fluorescence at 556 nm following 1 hour |
| | incubation |
| No DNA | No increase in fluorescence following 3 hour incubation |

The experiment demonstrated the capacity to simultaneously amplify and detect two unrelated target DNA sequences using amplification combined with detection via coamplified catalytic nucleic acid activity.

### Example 9

### A Simple Dipstick Test Employing the Methods Provided to Detect One or More Target Sequences

The schematic in FIG. 3 depicts a generalised format of an example of a multiplex “Dipstick” test kit as a useful embodiment. A multiplex reaction can simultaneously amplify several targets of interest (step i). If the reaction tube remains translucent to the naked eye, this indicates no amplification has occurred (negative result for all targets). If the reaction tube becomes turbid or cloudy, this indicates successful amplification (positive result for one or more targets). The exact species present in the positive sample can then be identified by exposure to the dipstick (step ii). In this example, five dual labeled fluorescent substrates are covalently attached at discrete locations to the dipstick (step iii). Five substrates would allow for the identification one positive and one negative amplification control and three targets of interest. While the sequences of each substrate must be distinct from each other, the fluorophore/quencher dye pair may be the same. Active DNAzymes generated during amplification from each target present in the sample will cleave the corresponding substrate. Cleavage of each fluorescent substrate will result in removal of the quencher and fluorescence at a specified location (step iv). The resulting banding pattern on the strip test could, for example, either identify the target species in the test sample, or the indication for personalised therapy. This reaction could be performed under isothermal conditions at any temperature suitable for use herein.

### Example 10

### A Simple Strip Test Employing the Methods Provided to Detect One or More Target Sequences

The schematic in FIG. 4 depicts further exemplary embodiments. For example, a “Dipstick test”, as described in FIG. 3 can be extended to a “Striptest” (a). In this embodiment, multiplex amplification can amplify a panel of samples (step i), of which the positive samples are further analysed with the Striptest (step ii), which contains covalently-attached, single colour capture substrates, specific to each amplicon in discrete locations. Target amplification results in substrate cleavage and the signal generation at the corresponding position (step iii). The resulting banding pattern could identify, for example, specific targets in a panel of patient samples, such as viral screening of blood products.

Multiplex amplification and detection or quantification can also be carried out in a homogeneous single tube format “Microtiter/well format” (b). Amplicons from each target amplified from a multiplex reaction harbour specific DNAzyme tag, which cleaves complementary substrate (Sub) labelled with distinct fluorophore (F) (step i). Successful target amplification from a multiplex amplification reaction can be determined by the wavelength of the signal generated by specific substrate cleavage at the end of the reaction. The change in fluorescence from these multiplexed reactions can be monitored by end point or in real time (step ii).

### Example 11

### Amplification and Detection in the Presence and Absence of “Outer” Primers

In this example, amplification reactions were carried out in either the presence or the absence of the outer primers. The inner L-FIP primer, inner L-BIP primer, L-Loop F primer, L-Loop B primer, L-cDzX/Loop B primer, outer L-F3 primer, outer L-B3 primer sequences and Sub X sequences were as described in Example 1.

Reactions contained lambda DNA (5 pg), 0.8 μM of L-FIP, 0.8 μM of L-BIP, 0.4 μM L-Loop F, 0.2 μM L-Loop B, 0.2 μM L-cDzX/Loop B, 0.4 μM of Sub X, 0.4 mM dNTPs (each of dATP, dCTP, dTTP, and dGTP), 0.8× thermopol buffer (16 mM Tris pH 8.8 at 25° C., 8 mM KCl, 1.6 mM MgSO₄, 0.08% Triton and 8 mM NH₄SO₂), 0.2×NEB3 buffer (2 mM Tris-HCl pH 7.9 at 25° C., 0.4 mM MgCl₂, 4 mM NaCl, 0.04 mM dithiolthreitol)), 1 mM additional MgSO₄, 1×ROX, and 8 units of Bst DNA polymerase (New England Biolabs) in a total reaction volume of 25 μl. Each reaction contained the above reagents plus the following concentrations of the outer primers L-F3 and L-B3, as shown in Table 11-1.

TABLE 11-1

| Reaction Mix | [Outer L-F3 primer] | | [Outer L-B3 primer] | |
|---|---|---|---|---|
| Mix 1 | 0.2 | μM | 0.2 | μM |
| Mix 2 | 0.1 | μM | 0.1 | μM |
| Mix 3 | 0.05 | μM | 0.05 | μM |
| Mix 4 | 0 | μM | 0 | μM |

Each reaction type was performed in triplicate. Control reactions lacking lambda DNA contained either no outer primers or 0.2 μM of both the L-F3 and L-B3 outer primers. The reactions were placed in an ABI PRISM 7700 (Applied Biosystems) and incubated at 56° C. for 60 minutes. Fluorescence was measured throughout the 60 minute amplification.

An increase in fluorescence was observed in all reactions which contained lambda template. This fluorescence increase was not dependent on the presence of the outer L-F3 and L-B3 primers, although the time taken to reach a threshold level of fluorescence was influenced by the concentration of outer primers. Table 11-2 shows a summary of the results obtained.

TABLE 11-2

| Reaction | Lambda | Concentration of outer | Time (min) to reach |
|---|---|---|---|
| type | DNA | primers | threshold fluorescence |
| Mix 1 | Present | 0.2 μM F3 and 0.2 μM B3 | 39.7 |
| Mix 2 | Present | 0.1 μM F3 and 0.1 μM B3 | 38.9 |
| Mix 3 | Present | 0.05 μM F3 and | 38.9 |
| | | 0.05 μM B3 | |
| Mix 4 | Present | None (0 μM F3 and B3) | 44.9 |
| Control | Absent | 0.2 μM F3 and 0.2 μM B3 | No increase in |
| Mix 1 | | | fluorescence at 60 min |
| Control | Absent | None (0 μM F3 and B3) | No increase in |
| Mix 4 | | | fluorescence at 60 min |

*Baseline was monitored from 1 to 25 min, the Threshold was set at 0.3.

The experiment demonstrates that the reaction for the amplification and detection assays can be performed in the presence or absence of the outer primers with only minimal impact on the reaction speed.

### REFERENCES

Patent Literature
“Catalytic nucleic acid-based diagnostic methods”. (A. V. Todd, C. J. Fuery and M. Cairns, 1999) U.S. 60/079,651; PCT/IB99/00848; WO 99/50452
“Zymogenic nucleic acid detection methods and related kits”. (A. V. Todd, C. J. Fuery and M. Cairns, 1999). U.S. Pat. No. 6,140,055; U.S. Pat. No. 6,201,113; PCT/IB99/00754, WO 99/45146.
U.S. Pat. No. 4,683,202.
U.S. Pat. No. 4,683,195.
U.S. Pat. No. 4,000,159.
U.S. Pat. No. 4,965,188.
U.S. Pat. No. 5,176,995.
EP 1 020 534 A1 (Eiken 1999)
EP 1 275 715 A1 (Eiken 2000)
EP 1 327 679 A1 (Eiken 2000)
EP 1 333 089 A1 (Eiken 2000)
EPO 684 315 A1 (Becton Dickinson and Company)

Nonpatent Literature
Breaker, R. R. DNA enzymes (1997) Nature Biotechnology 15: 427431.
Breaker, R. R. and Joyce, G. (1994) A DNA enzyme that cleaves RNA. Chemistry and Biology 1:223-229.
Carmi, N., et al. (1996) In vitro selection of self-cleaving DNAs. Chemistry and Biology 3:1039-1046.
Chehab, F. F., Doherty, M., Cai, S. P., Kan, Y. W., Cooper, S. and Rubin, E. M. (1987) Detection of sickle cell anaemia and thalassaemias [letter] [published erratum appears in Nature 1987 Oct. 22-28;329(6141):678]. Nature, 329, 293-294.
Compton, J. (1991) Nucleic acid sequence-based amplification. Nature, 350, 91-92.
Cuenoud, B. and Szostak, J. W. (1995) A DNA metalloenzyme with DNA ligase activity. Nature 375:611-614.
Eigen, M. and Rigler, R. (1994) Sorting single molecules: Application to diagnostics and evolutionary biotechnology. PNAS 91:5740-5747.
Fahy, E., et al. (1991) Self-sustained sequence replication (3SR): An iso-thermal transcription-based amplification alternative to PCR. PCR Methods Appl 1: 25-33.
Haseloff J. and Gerlach, W. L. (1988) Simple RNA enzymes with new and highly specific endoribonuclease activities. Nature, 334, 585-591.
Heid, C. A., Stevens, J., Livak, K. J. and Williams, P. M. (1996) Real time quantitative PCR. Genome Res, 6, 986-994.
Illangasekare, M., Sanchez, Nickles, T., and Yarus, M. (1995) Aminoacyl-RNA synthesis catalyzed by an RNA. Science 267:643-647.
Impey, Helen L., Tanya L. Applegate, Margaret A. Haughton, Caroline J. Fuery, Judith E. King and Alison V. Todd. (2000) Factors that influence deoxyribozyme cleavage during PCR. Analytical Biochemistry 286: 300-303.
Iwamoto, T et al. Journal of Clinical Microbiology 41: 2616-2622
Jonas, V., et al. (1993) Detection and identification of Mycobacterium tuberculosis directly from sputum sediments by amplification of rRNA. Journal of Clinical Microbiology 31:2410-2416.
Kramvis, A., Bukofzer, S. and Kew (1996) Comparison of Hepatitis B virus DNA extractions from serum by the QIAamp blood kit, Genereleaser, and the Phenol-chloroform method. Journal of Clinical Microbiology 34: 2731-2733.
Lee, L. G., Connell, C. R. and Bloch, W. (1993) Allelic discrimination by nick-translation PCR with fluorogenic probes. Nucleic Acids Research 21:3761-3766.
Leone, G., van Schijndel, H., van Gemen, B., Kramer, F. R. and Schoen, C. D. (1998) Molecular beacon probes combined with amplification by NASBA enable homogeneous, real-time detection of RNA. Nucleic Acids Res, 26, 2150-2155.
Li, Y. and Sen, D. A. (1996) A catalytic DNA for porphorin metallation. Nature Struct. Biol. 3:734-747.
Lizardi, P. M., et al. (1998) Mutation detection and single-molecule using rolling-circle amplification Nature Genetics 19:225-232.
Lohse, P. A. and Szostak, J. W. (1996) Ribozyme-catalyzed amino-acid transfer reactions. Nature 381: 442-444.
Mori et al; (2004) J. Biochem Biophys Methods 59:145-147
Nagamine, N. et al. (2002) Accelerated reaction by loop-mediated isothermal amplification using loop primers. Molecular and Cellular Probes. 16, 223-229.
Nazarenko, I. A., et al. (1997) A closed tube format for amplification and detection of DNA based on energy transfer. Nucleic Acids Research 25: 2516-2521.
Notomi, T. et al. (2000) Loop-mediated isothermal amplification of DNA. Nucleic Acids Res. 28:e63
Perriman, R. and Gerlach, W. L. (1992) Extended target site specificity for a hammerhead ribozyme. Gene 113:157-163.
Raillard, S. A. and Joyce, G. F. (1996) Targeting sites within HIV-1 cDNA with a DNA cleaving ribozyme. Biochemistry 35:11693-11701.
Saiki, R. K., Scharf, S., Faloona, F., Mullis, K. B., Horn, G. T., Erlich, H. A. and Arnheim, N. (1985) Enzymatic amplification of beta-globin genomic sequences and restriction site analysis for diagnosis of sickle cell anemia. Science, 230, 1350-1354.
Sambrook, J., Fritsch, E. F. and Maniatis, T. (1989) Molecular Cloning: A Laboratory Manual. 2nd Ed., New York: Cold Spring Harbour Laboratory Press.
Santoro, S. W. and Joyce, G. (1997) A general purpose RNA-cleaving DNA enzyme. PNAS 94:4262-4266.
Singh, K. K., Hanne, A., and Krupp, G. (2004) Real time monitoring of RNA and DNA reactions by fluorescence detection. Methods Mol. Biol. 252:34-48.
Tarasow, T. M., Tarasow, S. L. and Eaton, B. E. (1997) RNA-catalyzed carbon-carbon bond formation. Nature 389: 54-57.

Todd, Alison V., Caroline J. Fuery, Helen L. Impey, Tanya L. Applegate and Margaret A. Haughton. (2000) DzyNA-PCR: Use of DNAzymes to detect and quantify nucleic acid in a fluorescent real time format. Clinical Chemistry 46:5 625-630.
Todd, A. V., Ireland, C. M., Radloff T. J., Kronenberg, H. and Iland, H. J. (1991) Analysis of N-ras gene mutations in acute myeloid leukemia by allele specific restriction analysis. Am J Hematol, 38, 207-213.
Tyagi, S. and Kramer, F. R. (1996) Molecular Beacons: Probes that fluoresce on hybridization. Nature Biotechnology 14: 303-308
Urdea, M. (1993) Synthesis and characterization of branched DNA (bDNA) for direct and quantitative detection of CMV, HBV, HCV and HIV. Clin. Chem. 39:725-726.
Vet, J, Van der Rijt, B, and Blom, H. J. (2002) Molecular beacons: colorful analysis of nucleic acids. Expert Rev. Mol. Diagn. 2(1), 89-99.
Walker, G. T., et al. (1992) Strand Displacement Amplification—an isothermal, in vitro DNA amplification technique. Nucleic Acids Res. 20:1691.
Wittwer, C. T., Herrmann, M. G., Moss, A. A. and Rasmussen, R. P. (1997) Continuous fluorescence monitoring of rapid cycle DNA amplification. Biotechniques, 22, 130-131, 134-138.
Yong, S. L., Thomas, R. J. S. and Phillips, W. A. (1995) Single-step direct PCR amplification from solid tissues. Nucleic Acids Research 23:1640.

1. A method for detecting the presence of a nucleic acid sequence in a sample, the method comprising:
(a) providing a primer mixture comprising:
(i) a pair of inner primers; or
(ii) a pair of inner primers and at least one outer primer; or
(iii) a pair of inner primers and at least one loop primer; or
(iv) a pair of inner primers, at least one outer primer, and at least one loop primer;
wherein the pair of inner primers comprises a forward inner primer and a backward inner primer, and each said inner primer comprises a first portion that hybridizes to a sense sequence of a target nucleic acid sequence, and a second portion that hybridizes to an antisense sequence of the target nucleic acid sequence;
wherein each said outer primer hybridizes to a portion of the target nucleic acid sequence;
wherein each said loop primer comprises a portion complementary to a single stranded loop region on an amplicon produced from the extension of the forward inner primer or the backward inner primer;
wherein at least one primer in said primer mixture comprises an antisense sequence of a catalytic nucleic acid such that a corresponding sense strand of said catalytic nucleic acid is incorporated in an amplicon produced during amplification of said target nucleic acid sequence;
wherein, when the primer mixture does not comprise any loop primers, an antisense sequence of a catalytic nucleic acid is positioned between the first and the second portion of one or both of the forward and backward inner primers; and
wherein, when the primer mixture comprises at least one loop primer an antisense sequence of a catalytic nucleic acid is positioned either between the first and the second portion of one or both of the forward or backward inner primer; at the 5′ end of one or more loop primers, or both;
(b) contacting the sample with the primer mixture under conditions permitting catalytic nucleic acid activity and target-dependent, primer-initiated, DNA polymerase-mediated nucleic acid amplification;
wherein the DNA polymerase has strand displacement activity;
(c) incubating the sample with the primer mixture to allow the primer mixture to initiate amplification, when the target nucleic acid sequence is present, to produce amplicons comprising the catalytic nucleic acid; and
(d) determining the presence of the catalytic nucleic acid activity, thereby determining the presence of target nucleic acid sequence in the sample.

2. The method of claim 1, further comprising the step of determining the amount of catalytic nucleic acid activity.

3. The method of claim 2 further comprising the step of comparing the amount of activity so determined to a known standard, thereby quantitatively determining the amount of the target nucleic acid sequence present in the sample.

4. The method of claim 1 wherein the catalytic nucleic acid is a DNAzyme.

5. The method of claim 4 wherein the DNAzyme is a 10:23 DNAzyme or an 8:17 DNAzyme.

6. The method of claim 1 wherein the catalytic nucleic acid is a ribozyme, and an RNA polymerase and promoter sequence therefor are included at least in the incubating step.

7. The method of claim 1 wherein the target nucleic acid sequence is DNA.

8. The method of claim 1 wherein the target nucleic acid sequence is RNA, and the method further comprises the step of reverse transcribing the sample prior to step (c).

9. The method of claim 1 wherein the catalytic nucleic acid activity comprises the detectable modification of a chemical substrate.

10. The method of claim 9 wherein the modification comprises formation or cleavage of one or more phosphodiester bonds, or ligation or cleavage of at least one nucleic acid.

11. The method of claim 9 wherein the substrate is a fluorescently-labeled nucleic acid molecule, and the modification is cleavage thereof.

12. The method of claim 9 wherein the substrate is a DNA/RNA chimera.

13. The method of claim 1 wherein the target nucleic acid sequence is from a human, a bacterium, a mycoplasma, an archaea, a plant, an animal, or a virus.

14. The method of any of claim 1 wherein the presence of the target nucleic acid sequence in the sample is indicative of a genetic disorder.

15. The method of claim 1 wherein the sample is a forensic sample, an environmental sample, an agricultural sample, or a veterinary sample.

16. The method of claim 1 wherein the primer-initiated nucleic acid amplification is LAMP.

17. The method of claim 1 wherein the incubation is at a temperature of about 37° C. to about 56° C.

18. The method of claim 1 wherein the primer mixture comprises a pair of inner primers but no outer primers or loop primers.

19. The method of claim 1 wherein the primer mixture comprises a pair of inner primers and at least one outer primer, but no loop primers.

20. The method of claim 1 wherein the primer mixture comprises a pair of inner primers and at least one loop primer, but no outer primers.

21. The method of claim 1 wherein the primer mixture comprises a pair of inner primers, at least one outer primer, and at least one loop primer.

22. A method of detecting the presence of each of a plurality of target nucleic acid sequences in a sample, the method comprising:
(a) providing a primer mixture comprising, for each of the plurality of target nucleic acid sequences to be detected at least:
(i) a pair of inner primers; or
(ii) a pair of inner primers and at least one outer primer; or
(iii) a pair of inner primers and at least one loop primer; or
(iv) a pair of inner primers, at least one outer primer, and at least one loop primer;
wherein each pair of inner primers comprises a forward inner primer and a backward inner primer, and each said inner primer comprises a first portion that hybridizes to a sense sequence of at least one of the plurality of target nucleic acid sequences, and a second portion that hybridizes to an antisense sequence of that target nucleic acid sequence;
wherein each said outer primer hybridizes to a portion of at least one of the plurality of target nucleic acid sequences;
wherein each said loop primer comprises a portion complementary to a single stranded loop region on an amplicon produced from the extension of at least one forward inner primer or backward inner primer corresponding to at least one of the plurality of target nucleic acid sequences;
wherein for each of the plurality of target nucleic acid sequences, at least one primer in said primer mixture comprises an antisense sequence of a distinctly detectable catalytic nucleic acid such that a corresponding sense strand of said distinctly detectable catalytic nucleic acid is incorporated in an amplicon produced during amplification of that target nucleic acid sequence;
wherein for each of the plurality of target nucleic acid sequences, when the primer mixture does not comprise any loop primers for that target nucleic acid sequence, said antisense sequence of a distinctly detectable catalytic nucleic acid is positioned between the first and the second portion of one or both of the forward or backward inner primers; and
wherein for each of the plurality of target nucleic acid sequences, when the primer mixture comprises at least one loop primer for that target nucleic acid sequence, the antisense sequence of a distinctly detectable catalytic nucleic acid is positioned between the first and the second portion of one or both of the forward or backward inner primers, or at the 5′ end of one or more loop primers, or both;
(b) contacting the sample with the primer mixture under conditions permitting catalytic nucleic acid activity and target sequence-dependent, primer-initiated, DNA polymerase-mediated nucleic acid amplification;
wherein the DNA polymerase has strand displacement activity;
(c) incubating the sample with the primer mixture to allow the primer mixture to initiate amplification of each of the plurality of target nucleic acid sequences, when that target nucleic acid sequence is present, to produce amplicons comprising the distinctly detectable catalytic nucleic acid; and
(d) determining the presence of each of the uniquely detectable catalytic nucleic acid activities, thereby determining the presence of the corresponding target nucleic acid sequence in the sample.

23. The method of claim 22 further comprising the step of determining the amount of at least one distinctly detectable catalytic nucleic acid activity.

24. The method of claim 23 further comprising the step of comparing the amount of each activity so determined to a known standard for that activity, thereby quantitatively determining the amount of each corresponding target nucleic acid sequence present in the sample.

25. The method of claim 22 wherein at least one catalytic nucleic acid is a DNAzyme.

26. The method of claim 25 wherein the DNAzyme is a 10:23 DNAzyme or an 8:17 DNAzyme.

27. The method of claim 22 wherein at least one catalytic nucleic acid is a ribozyme, and an RNA polymerase and promoter sequence therefor are included at least in the incubating step

28. The method of claim 22 wherein at least one of the plurality of target nucleic acid sequences is RNA, and the method further comprises the step of reverse transcribing the sample prior to step (c).

29. The method of claim 22 wherein the catalytic nucleic acid activity comprises the detectable modification of a chemical substrate.

30. The method of claim 29 wherein the substrate is a fluorescently-labeled nucleic acid molecule, and the modification is cleavage thereof.

31. The method of claim 29 wherein the substrate is a DNA/RNA chimera.

32. The method of claim 22 wherein one or more of the plurality of target nucleic acid sequences are from a human, a bacterium, a mycoplasma, an archaea, a plant, an animal, or a virus.

33. The method of claim 22 wherein the presence of at least one of the plurality of target nucleic acid sequences in the sample is indicative of a genetic disorder.

34. The method of claim 22 wherein the sample is a forensic sample, an environmental sample, an agricultural sample, or a veterinary sample.

35. The method of claim 22 wherein the primer-initiated nucleic acid amplification is LAMP.

36. The method of claim 22 wherein the incubation is at a temperature of about 37° C. to about 56° C.

37. The method of claim 22 wherein the method is conducted under substantially isothermal conditions.

38. The method of claim 22 wherein the primer mixture comprises a pair of inner primers, but no outer primers or loop primers.

39. The method of claim 22 wherein the primer mixture comprises a pair of inner primers and at least one outer primer, but no loop primers.

40. The method of claim 22 wherein the primer mixture comprises a pair of inner primers and at least one loop primer, but no outer primers.

41. The method of claim 22 wherein the primer mixture comprises a pair of inner primers, at least one outer primer, and at least one loop primer.

42. A method of detecting the presence of any of a plurality of target nucleic acid sequences in a sample, the method comprising:
(a) providing a primer mixture comprising one or more primers sufficient for amplifying each of the plurality of target nucleic acid sequences to be detected;
wherein for each of the plurality of target nucleic acid sequences, there is at least one primer in said primer mixture comprising an antisense sequence of a catalytic nucleic acid such that a corresponding sense strand of said catalytic nucleic acid is incorporated into an amplicon produced during amplification of that target nucleic acid sequence;
(b) contacting the sample with the primer mixture under conditions permitting catalytic nucleic acid activity and target sequence-dependent, primer-initiated, DNA polymerase-mediated nucleic acid amplification;
(c) incubating the sample with the primer mixture to allow the primer mixture to initiate amplification of any of the plurality of target nucleic acid sequences, when that target nucleic acid sequence is present, to produce amplicons comprising the catalytic nucleic acid; and
(d) determining the presence of the catalytic nucleic acid activity from an amplicon produced during the amplification of any of the target nucleic acid sequences, thereby determining the presence of any of the target nucleic acid sequences in the sample.

43. The method of claim 42 wherein the DNA polymerase has strand displacement activity, and wherein the primer mixture comprises at least:
(i) a pair of inner primers; or
(ii) a pair of inner primers and at least one outer primer; or
(iii) a pair of inner primers and at least one loop primer; or
(iv) a pair of inner primers, at least one outer primer, and at least one loop primer;
wherein each pair of inner primers comprises a forward inner primer and a backward inner primer, and each said inner primer comprises a first portion that hybridizes to a sense sequence of at least one of the plurality of target nucleic acid sequences, and a second portion that hybridizes to an antisense sequence of that target nucleic acid sequence;
wherein each said outer primer hybridizes to a portion of at least one of the plurality of target nucleic acid sequences;
wherein each said loop primer comprises a portion complementary to a single stranded loop region on an amplicon produced from the extension of at least one forward inner primer or backward inner primer corresponding to at least one of the plurality of target nucleic acid sequences;
wherein for each of the plurality of target nucleic acid sequences, when the primer mixture does not comprise any loop primers for that target nucleic acid sequence, said antisense sequence of a detectable catalytic nucleic acid is positioned between the first and the second portion of one or both of the forward and backward inner primers; and
wherein for each of the plurality of target nucleic acid sequences, when the primer mixture comprises at least one loop primer for that target nucleic acid sequence, the antisense sequence of a detectable catalytic nucleic acid is positioned between the first and the second portion of one or both of the forward or backward inner primer, or at the 5′ end of one or more loop primers, or both positions.

44. The method of claim 42, further comprising the step of determining the total amount of catalytic nucleic acid activity.

45. The method of claim 42 wherein at least one catalytic nucleic acid is a DNAzyme.

46. The method of claim 45 wherein the DNAzyme is a 10:23 DNAzyme or an 8:17 DNAzyme.

47. The method of claim 42 wherein at least one catalytic nucleic acid is a ribozyme, and an RNA polymerase and promoter sequence therefor are included at least in the incubating step.

48. The method of claim 42 wherein at least one of the plurality of target nucleic acid sequences is RNA, and the method further comprises the step of reverse transcribing the sample prior to step (c).

49. The method of claim 42 wherein the catalytic nucleic acid activity comprises the detectable modification of a chemical substrate which is a fluorescently-labeled nucleic acid molecule, and the modification is cleavage thereof.

50. The method of claim 42 wherein the catalytic nucleic acid activity modifies a DNA/RNA chimera substrate.

51. The method of claim 42 wherein one or more of the target nucleic acid sequences are from a human, a bacterium, a mycoplasma, an archaea, a plant, an animal, or a virus.

52. The method of claim 42 wherein the presence or absence in the sample of any of the target nucleic acid sequences is indicative of a genetic condition, a disease condition, or an infection.

53. The method of claim 42 wherein the sample is a forensic sample, an environmental sample, an agricultural sample, or a veterinary sample.

54. The method of claim 42 wherein the nucleic acid amplification method is PCR, SDA, RCA, LAMP, TMA, 3SR, or NASBA.

55. The method of claim 42 wherein the incubation is conducted at a temperature of about 37° C. to about 56° C.

56. The method of claim 42 wherein the primer mixture comprises a pair of inner primers, but no outer primers or loop primers.

57. The method of claim 42 wherein the primer mixture comprises a pair of inner primers and at least one outer primer, but no loop primers.

58. The method of claim 42 wherein the primer mixture comprises a pair of inner primers and at least one loop primer, but no outer primers.

59. The method of claim 42 wherein the primer mixture comprises a pair of inner primers, at least one outer primer, and at least one loop primer.

60. The method of claim 42 wherein the presence or absence in the sample of any of the target nucleic acid sequences is indicative of a bacterium, a virus, an insect, or a genetically-modified organism.

61. A device for detecting the presence, in a sample placed therein, of at least one target nucleic acid sequence, the device comprising:
a reaction vessel into which the sample is introduced, said reaction vessel comprising a reaction mixture suitable for target sequence-dependent, primer-initiated, DNA polymerase-mediated nucleic acid amplification under conditions also permitting catalytic nucleic acid activity,
the reaction mixture comprising the reactants for amplification of nucleic acids in the sample and a primer mixture comprising one or more primers sufficient for amplifying each of the at least one target nucleic acid sequences to be detected;
wherein for each of the at least one target nucleic acid sequences to be detected, there is at least one primer in said primer mixture comprising an antisense sequence of a catalytic nucleic acid such that a corresponding sense strand of said catalytic nucleic acid is incorporated into an amplicon produced when that target is present in the sample; said sense strand comprising an active catalytic nucleic acid that recognizes and modifies a corresponding substrate;
a support means for bearing the substrate for each catalytic nucleic acid activity corresponding to each of the at least one target nucleic acid sequences to be detected; wherein each such substrate produces a detectable signal upon modification thereof by the catalytic nucleic acid.

62. The device of claim 61 for detecting the presence, in a sample placed therein, of each of a plurality of target nucleic acid sequences.

63. The device of claim 61 wherein each substrate is localized in a discrete location on the support means, and the detectable signal remains so localized during detection.

64. The device of claim 61 wherein each substrate is covalently localized and the detectable signal remains covalently attached to the support means for detection after modification thereof.

65. The device of claim 61 wherein the substrate is cleaved by the catalytic nucleic acid.

66. The device of claim 61 wherein each detectable signal is distinct.

67. The device of claim 61 wherein the detectable signal produced from each substrate is not distinct, and the device detects the presence of any of a plurality of target nucleic acids.

68. The device of claim 61 wherein the support means is in the form of a dipstick that can be at least partially inserted into the reaction vessel.

69. The device of claim 61 further comprising a negative control reaction, and a positive control reaction.

70. The device of claim 61 wherein the detectable signal comprises a colorometric signal, fluorescence, luminescence, turbidity, or radioactivity.

71. The device of claim 61 wherein the nucleic acid amplification method is PCR, SDA, RCA, LAMP, TMA, 3SR, or NASBA.

72. The device of claim 61 that can be incubated isothermally after the sample is added at a temperature of about 37° C. to about 65+ C.

73. The device of claim 61 in which a change in signal is monitored in real-time.

74. The device of claim 61 that can be conducted under field conditions, in an office, or in a mobile laboratory.

75. A kit for use in detecting the presence of a target nucleic acid sequence in a sample comprising:
(a) a primer mixture comprising:
(i) a pair of inner primers; or
(ii) a pair of inner primers and at least one outer primer; or
(iii) a pair of inner primers and at least one loop primer; or
(iv) a pair of inner primers, at least one outer primer, and at least one loop primer;
wherein the pair of inner primers comprises a forward inner primer and a backward inner primer, and each said inner primer comprises a first portion that hybridizes to a sense sequence of a target nucleic acid sequence, and a second portion that hybridizes to an antisense sequence of the target nucleic acid sequence;
wherein each said outer primer present hybridizes to a portion of the target nucleic acid sequence;
wherein each said loop primer present comprises a portion complementary to a single stranded loop region on an amplicon produced from the extension of the forward inner primer or the backward inner primer;
wherein at least one primer in said primer mixture comprises an antisense sequence of a catalytic nucleic acid such that a corresponding sense strand of said catalytic nucleic acid is incorporated in an amplicon produced during amplification of said target nucleic acid;
wherein, when the primer mixture does not comprise any loop primers, said antisense sequence of a catalytic nucleic acid is positioned between the first and the second portion of one or both of the forward or backward inner primers; and
wherein, when the primer mixture comprises at least one loop primer, the antisense sequence of a catalytic nucleic acid is positioned between the first and the second portion of one or both of the forward or backward inner primer, or at the 5′ end of one or more of the loop primers, or both; and
(b) a substrate modifiable by the catalytic nucleic acid and whose modification generates a detectable signal;
(c) a reaction mixture providing conditions permitting catalytic nucleic acid activity and target-dependent, primer-initiated, DNA polymerase-mediated nucleic acid amplification, and reactants required therefor; and
(d) a DNA polymerase having strand displacement activity.

76. The kit of claim 75 wherein the primer mixture comprises a pair of inner primers, but no outer primers or loop primers.

77. The kit of claim 75 wherein the primer mixture comprises a pair of inner primers and at least one outer primer, but no loop primers.

78. The kit of claim 75 wherein the primer mixture comprises a pair of inner primers and at least one loop primer, but no outer primers.

79. The kit of claim 75 wherein the primer mixture comprises a pair of inner primers, at least one outer primer, and at least one loop primer.

80. The kit of claim 75 further comprising instructions for detecting the target nucleic acid.

81. The kit of claim 75 further comprising a reverse transcriptase and reagents for producing a DNA from an RNA target nucleic acid.

82. A kit for use in detecting the presence of each of a plurality of target nucleic acid sequences in a sample, the kit comprising:
(a) a primer mixture comprising, for each of the plurality of target nucleic acid sequences to be detected at least:
(i) a pair of inner primers; or
(ii) a pair of inner primers and at least one outer primer; or
(iii) a pair of inner primers and at least one loop primer; or
(iv) a pair of inner primers, at least one outer primer, and at least one loop primer;
wherein each pair of inner primers comprises a forward inner primer and a backward inner primer, and each said inner primer comprises a first portion that hybridizes to a sense sequence of at least one of the plurality of target nucleic acid sequences, and a second portion that hybridizes to an antisense sequence of that target nucleic acid sequence;
wherein each said outer primer hybridizes to a portion of at least one of the plurality of target nucleic acid sequences;
wherein each said loop primer comprises a portion complementary to a single stranded loop region on an amplicon produced from the extension of at least one forward inner primer or backward inner primer corresponding to at least one of the plurality of target nucleic acid sequences;
wherein for each of the plurality of target nucleic acid sequences, at least one primer in said primer mixture comprises an antisense sequence of a distinctly detectable catalytic nucleic acid such that a corresponding sense strand of said distinctly detectable catalytic nucleic acid is incorporated in an amplicon produced during amplification of that target nucleic acid sequence;
wherein for each of the plurality of target nucleic acid sequences, when the primer mixture does not comprise any loop primers for that target nucleic acid sequence, said antisense sequence of a distinctly detectable catalytic nucleic acid is positioned between the first and the second portion of one or both of the forward or backward inner primer; and
wherein for each of the plurality of target nucleic acid sequences, when the primer mixture comprises at least one loop primer for that target nucleic acid sequence, the antisense sequence of a distinctly detectable catalytic nucleic acid is positioned between the first and the second portion of one or both of the forward or backward inner primer, or at the 5′ end of one or more loop primers, or both;
(b) for each of the plurality of target nucleic acid sequences, a substrate modifiable by the catalytic nucleic acid corresponding to that target nucleic acid sequence, the modification of which substrate generates a distinctly detectable signal;
(c) a reaction mixture providing conditions permitting catalytic nucleic acid activity and target-dependent, primer-initiated, DNA polymerase-mediated nucleic acid amplification, and reactants required therefor; and
(d) a DNA polymerase having strand displacement activity.

83. The kit of claim 82 wherein the primer mixture comprises a pair of inner primers, but no outer primers or loop primers.

84. The kit of claim 82 wherein the primer mixture comprises a pair of inner primers and at least one outer primer, but no loop primers.

85. The kit of claim 82 wherein the primer mixture comprises a pair of inner primers and at least one loop primer, but no outer primers.

86. The kit of claim 82 wherein the primer mixture comprises a pair of inner primers, at least one outer primer, and at least one loop primer.

87. The kit of claim 82 further comprising instructions for detecting the target nucleic acid.

88. The kit of claim 82 further comprising a reverse transcriptase and reagents for producing a DNA from an RNA target nucleic acid, or both.

89. A kit for use in detecting the presence of any of a plurality of target nucleic acid sequences in a sample, the kit comprising:
(a) a primer mixture comprising one or more primers sufficient for amplifying each of the plurality of target nucleic acid sequences to be detected;
wherein for each of the plurality of target nucleic acid sequences, there is at least one primer in said primer mixture comprising an antisense sequence of a catalytic nucleic acid such that a corresponding sense strand of said catalytic nucleic acid is incorporated into an amplicon produced during amplification of that target nucleic acid sequence;
(b) for each of the plurality of target nucleic acid sequences, a substrate modifiable by the catalytic nucleic acid corresponding to that target nucleic acid sequence, the modification of which substrate generates a detectable signal;
(c) a reaction mixture providing conditions permitting catalytic nucleic acid activity and target-dependent, primer-initiated, DNA polymerase-mediated nucleic acid amplification, and reactants required therefor; and
(d) a DNA polymerase suitable for amplifying the target nucleic acid sequences.

90. The kit of claim 89 wherein the DNA polymerase has strand displacement activity, and wherein the primer mixture comprises at least:
(i) a pair of inner primers; or
(ii) a pair of inner primers and at least one outer primer; or
(iii) a pair of inner primers and at least one loop primer; or
(iv) a pair of inner primers, at least one outer primer, and at least one loop primer;
wherein each pair of inner primers comprises a forward inner primer and a backward inner primer, and each said inner primer comprises a first portion that hybridizes to a sense sequence of at least one of the plurality of target nucleic acid sequence, and a second portion that hybridizes to an antisense sequence of that target nucleic acid sequence;
wherein each said outer primer hybridizes to a portion of at least one of the plurality of target nucleic acid sequences;
wherein each said loop primer comprises a portion complementary to a single stranded loop region on an amplicon produced from the extension of at least one forward inner primer or backward inner primer corresponding to at least one of the plurality of target nucleic acid sequences;
wherein for each of the plurality of target nucleic acid sequences, when the primer mixture does not comprise any loop primers for that target nucleic acid sequence, said antisense sequence of a detectable catalytic nucleic acid is positioned between the first and the second portion of one or both of the forward and backward inner primers; and wherein for each of the plurality of target nucleic acid sequences, when the primer mixture comprises at least one loop primer for that target nucleic acid sequence, the antisense sequence of a detectable catalytic nucleic acid is positioned between the first and the second portion of one or both of the forward and backward inner primers, or at the 5′ end of one or more loop primers, or both.

91. The kit of claim 90 wherein the nucleic acid amplification method is PCR, SDA, RCA, LAMP, TMA, 3SR, or NASBA.

92. The kit of claim 90 further comprising instructions for detecting the target nucleic acid.

93. The kit of claim 90 further comprising a reverse transcriptase and reagents for producing a DNA from an RNA target nucleic acid, or both.

94. A DNA molecule comprising at least a first portion complementary to at least a first portion of a target nucleic acid sequence, a second portion complementary to an antisense sequence of a second portion of the target nucleic acid sequence, and a third portion comprising an antisense sequence of a catalytic nucleic acid; said third portion positioned between the first and second portions of said DNA molecule.

95. A method for the amplification and detection of at least one target nucleic acid sequence comprising using the molecule of claim 94 as a primer during the amplification of the target nucleic acid sequence, wherein at least one amplicon produced during amplification comprises the sense strand of the catalytic nucleic acid, and wherein the detection comprises the modification of at least one detectable substrate by the catalytic nucleic acid in the at least one amplicon.

96. The method of claim 95 wherein the amplification is isothermal and conducted at a temperature less than about 62° C.

97. The method of claim 95 wherein the amplification comprises the use of a DNA polymerase with strand displacement activity.

98. The method of claim 95 wherein the target nucleic acid is RNA and the method comprises the additional step of reverse transcribing the RNA into DNA prior to amplification.

99. The method of claim 95 wherein the modification of the substrate is cleavage, and the method further comprises the step of using a plurality of cleavable substrates, the cleavage of each of which is distinctly detectable, wherein there is one such substrate for each of a plurality of target nucleic acid sequences.

100. The method of any of claims 1, 22, 42, or 95 wherein one or more primers comprise at least one backbone modification that comprises a blocking substituent to block copying of one or more portions of the primer.

101. The method of claim 100 wherein the blocking substituent is HEG.

102. A kit according to any of claims 75, 82, or 89 wherein the primer mixture comprises one or more primers comprising at least one backbone modification that comprises a blocking substituent to block copying of one or more portions of the primer.

103. The kit of claim 102 wherein the blocking substituent is HEG.

104. The DNA molecule of claim 94 further comprising a backbone modification selected from a hexethylene glycol monomer substituent or 2-O-alkyl RNA substituent.
